(19) United States
(12) Patent Application Publication
(10) Pub. No.: US 2017/0252200 A1
(43) Pub. Date: Sep. 7, 2017

(54) A ONE-SIZE-FITS ALL UNLOADING AND/OR STABILIZING KNEE BRACE FOR USE FROM EITHER THE INSIDE OR THE OUTSIDE OF THE LEFT OR RIGHT KNEE

(71) Applicant: Scott Andrew Taylor, Woodstock, GA (US)

(72) Inventor: Scott Andrew Taylor, Woodstock, GA (US)

(21) Appl. No.: 15/316,924

(22) PCT Filed: Jun. 13, 2016
(86) PCT No.: PCT/US16/37289

§ 371 Date: Dec. 7, 2016

Related U.S. Application Data
(60) Provisional application No. 62/175,215, filed on Jun. 12, 2015.

(51) Int. Cl. A61F 5/34; A61F 5/01

(52) U.S. Cl. CPC A61F 5/34; A61F 5/0123; A61F 2005/0174

(57) ABSTRACT

A knee brace of near universal application for any patient needing left or right knee stabilization, and/or suffering from arthritis, bone on bone contact, and/or narrowed joint spacing in either left or right knee. Brace is mountable on outside or inside of left or right knee. Brace includes a selectively activated force device for exerting pressure directly by pushing at a joint line of a knee or indirectly by pulling on a knee strap wrapped around knee and force device. A base, adjacent to the force device, at least partially resists pressure of force device, and is adjacent a flexor that allows knee flexing when brace is worn. Extensions connect flexor with base and force device to thigh and calf attachment straps. A knee strap may be used with force device. Knee strap has opening to fit over patella and straps for wrapping around knee without hampering knee flexion.

### CROSS-REFERENCE TO RELATED APPLICATION

This application claims priority to and benefit of the prior filed co-pending and commonly owned provisional application filed in the United States Patent and Trademark Office on Jun. 12, 2015, assigned Ser. No. 62/175,215, entitled A Knee Brace, a Kit for a Knee Brace, and Systems and Methods of Using Same, and incorporated herein by reference.

### FIELD OF THE INVENTION

The invention relates to apparatuses, devices, kits, and systems and methods of using same with respect to orthopedic devices such as knee braces, and particularly, to knee braces for unloading stress from affected joints, for stabilizing affected joints, and/or for other issues.

### BACKGROUND

The knee is a joint with three parts. The knee joint is formed by the meeting of the thigh bone (femur) and the large shin bone (tibia). This joint has a medial (inner) compartment and a lateral (outer) compartment. The knee cap (patella) joins the femur to form a third joint, which is called the patellofemoral joint. The patella protects the front of the knee joint.

FIG. 1 illustrates the fronts of a person's left and right knee joints. The lateral (outer) compartment of the left knee joint is circled and identified. The arrows within the circled lateral (outer) compartment of knee point out the space within the lateral (outer) compartment. The medial (inner) compartment of the left knee joint also is circled and labeled separately from the lateral (outer) compartment. The arrows inside the circle identifying the medial (inner) compartment show reduced spacing or bone on bone contact instead of a normal space. The bone on bone contact may be painful to the person. The person has the same pain issue in his or her right knee because the medial (inner) compartment of the right knee also has bone on bone contact. The pain may be reduced through the use of an “unloader” knee brace on each knee. The unloader knee brace strives to apply pressure on the side of the knee at the joint line to create a slight biomechanical change to the afflicted knee to restore some joint space or reduce pressure in the affected area.

The unloader knee brace also may be used in the same general manner to reduce the pain of a patient who suffers such pain from “compromised” or “narrowed” joint spacing.

The term “medial (inner) compartment” is used interchangeably herein with the term “medial joint compartment.” The term “lateral (outer) compartment” is used interchangeably herein with the term “lateral joint compartment.” All of these four terms refer to interior elements of a knee.

### SUMMARY

The knee brace of the invention offers many advantages, some of which are mentioned herein. The design of the inventive knee brace may be considered simple, elegant, and versatile. Embodiments of the invention may be made universal in sizing. The same knee brace may be used for either the right knee or the left knee of a patient. The same knee brace also may be used without modification to create varus or valgus stress for unloading and manipulation of the knee. With only minor modifications (as are described below), the same knee brace may be made to utilize different pull force mechanics to achieve the same unloading and manipulation effect. The knee brace may be used for unloading either the medial (inner) or the lateral (outer) compartment of either the right knee or the left knee for almost any sized patient.

Advantageously, the knee brace of the invention may be used in other ways than mentioned in the previous paragraph. For example, the knee brace may be used for general knee instability problems, proprioception issues, and post-operative applications. In these cases, the force device of the knee brace may be passive except to create an intimate fit to the patient's leg. Such uses of the knee brace may assist a patient for ambulation, stability, and/or range of motion control instead of or in addition to using the knee brace for unloading the knee. Such other uses of the knee brace may demonstrate its versatility, and encourage manufacturers, clinicians, patients and/or others to make use of the knee brace in ways in addition and/or in the alternative to those described herein.

A user may benefit from the advantages of the knee brace of the invention especially because of its simple, versatile, and elegant design. The user may be readily fitted for the knee brace by a clinician at the time of the patient's visit without measuring or with minimal measuring. The user may be readily fitted for the knee brace by a clinician without him or her having to order a specific brace particularly sized for the patient's leg and appropriate to his or her condition. The user may only need minimal training whether the user is to use the knee brace for either the right or left knee, and/or whether the user is to use the knee brace for the inside or outside of the knee or whether the user is to use the knee brace with a push or a pull force mechanism. The user may find the knee brace easy to use whether the user is handling the knee brace, putting the knee brace on, wearing it, and/or taking it off. The user may not have to return or at least not have to return as often to the clinician for callbacks and troubleshooting regarding the exemplary knee brace as may be the case with other knee braces.

A user may benefit from the advantage of the knee brace of the invention being light in weight. Its light-weight as well as its simple, versatile, and elegant design may contribute to making the knee brace easy to use.

A user also may benefit from the low profile of the knee brace. A low profile may make the knee brace easier to use and easier to wear initially when the patient is first beginning to use the knee brace, and/or may allow the user to wear the knee brace for longer periods after the initial acclimatization than some prior art knee braces.

The low profile also may be advantageous to a user because the knee brace does not extend out much from the user's leg when compared to some other knee braces. A user may wear the low profile knee brace more comfortably than other knee braces. For example, the exemplary knee brace may be worn under some styles of slacks or pants because of its low profile. A user inclined to wear skirts or shorts may find that the low profile of the exemplary knee brace does not interfere with those fashions as much as some other knee braces.

A welcome advantage of the knee brace to a user may be that it may be relatively inexpensive at least compared to other knee braces. For example, the invention may be implemented in a very basic embodiment such as a knee brace including a simple hinge with no range of motion control features. The hinge also may be referred to herein as a “knee flexor” in that it may allow a patient to at least partially flex his or her knee when the knee brace is in use. This basic example of the inventive knee brace may be made to be bought and sold without a prescription over the Internet and/or in a sporting goods or retail store. The basic knee brace may be offered at a low price point, which may please a patient as well as others.

The simple, versatile and elegant design of the knee brace of the invention may offer advantages to the clinician. The knee brace may be easily fitted to a patient or user. The clinician may require only minimal training in learning to apply the knee brace. The clinician may use the same knee brace unit whether applying the knee brace to the user's right knee or the left knee, and whether applying the knee brace to the inside or the outside of the knee of the user. The clinician may use the same knee brace with or without minor modifications whether the knee brace is to be used to unload either the medial (inner) or the lateral (outer) compartment of either the user's right or left knee. The clinician may be able to vary the force from a push to pull configuration to achieve best patient outcome based at least on a patient's preference, anatomy, effectiveness, etc.

As noted previously, the knee brace may be used for reasons other than or in addition to the unloading of a patient's knee. For example, an exemplary knee brace may be used for general support and stability by using the force device, push or pull, passively, or just for fitting the brace intimately, but not engaging the force properties of the mechanism of the knee brace. These other uses of the knee brace may be welcomed as advantages by clinicians and others.

The clinician also may find the simple, versatile, and elegant design of the knee brace of the invention an advantage in many ways. For example, an embodiment of the knee brace may be universal in sizing. This exemplary knee brace of universal sizing may be used by the clinician on patients of varying shapes and sizes including those with different leg lengths, angles and girths.

A knee brace according to the invention of universal sizing may offer benefits to a clinician in addition to those previously mentioned as being usable on patients of varying shapes and sizes. For example, if a clinician makes use of the invention of universal sizing for his or her patients, the clinician may be able to rid his or her inventory of the myriad knee braces of different sizes, shapes, features previously necessary to accommodate different patients and their respective needs. The clinician who makes use of the invention of universal sizing may have a more manageable inventory because he or she only has to stock embodiments of the invention of universal sizing. In addition, the knee brace of universal sizing may eliminate or significantly reduce the need for custom-made knee braces.

As another example, the clinician may find the simple, versatile, and elegant design of the knee brace an advantage in working with a patient such as in applying the knee brace, adjusting it, wearing it, and/or removing it. The clinician may benefit by having fewer patient callbacks and follow up trouble-shooting visits with a patient using a knee brace of the invention. The clinician also may be pleased with respect to the knee brace because of a patient's satisfaction in using the knee brace and in its cost.

Other advantages of the knee brace of the invention may include the fact that its simple, versatile, and elegant design may be relatively easy and inexpensive to manufacture and sell. For example, an embodiment of the knee brace may be made available elegant design may be relatively easy and inexpensive to manufacture and sell. For example, an embodiment of the knee brace may be made available “over the counter” to a user without his or her need for a prescription and/or other authorization from a clinician and/or other authority.

Yet another advantage of the knee brace of the invention is that it may be used with one or more other therapeutic and/or other devices relating to the patient's knee and/or otherwise. For example, an embodiment of the knee brace may include one or more features to facilitate the use of one or more other devices. As a particular example, the exemplary knee brace may include a hook and/or other attachment means or mechanism by which one or more other therapeutic (and/or other) devices may be attached to and used with the exemplary knee brace. As another particular example, the exemplary knee brace may include a “pocket” into which one or more therapeutic (and/or other) devices may be placed for use with the knee brace and/or to be carried by it in the pocket. Embodiments of the knee brace may be compatible by inclusion of one or more features for attachment with one or more devices that may provide transcutaneous electrical nerve stimulation (TENS), neuromuscular electrical nerve stimulation (NMES), pulsed electromagnetic frequency (PMEF) and/or the like and/or other. When a knee brace according to the invention includes one or more additional devices, the knee brace also may carry out its unloading functionality or the knee brace may simply be used to carry the one or more additional devices. Further, the knee brace may be used with one or more additional devices and/or features whenever the knee brace may be used besides those times mentioned previously in this paragraph, such as for example, but limited to, use of the knee brace for instability, range of motion control, etc.

A prime use of the knee brace of the invention may be to provide pain relief to a user suffering from osteoarthritis in the knee. As an example, the knee brace may provide pain relief to a user suffering from compartment knee osteoarthritis (such as for example, but not limited to, affecting the inner and/or outer portion of the knee, and/or behind the kneecap/patella).

The invention, however, is not to be limited to use as a pain reliever only. The invention also may be used for knee preservation. Embodiments of the invention may be used with fractures to provide stability and offloading for healing. In addition, embodiments of the invention may be used for the condition of osteochodritis dissecans (OCD) in juvenile orthopedics. Embodiments of the invention may be used with common meniscus injuries to offload for pain management or healing. Further, the invention may be used as or in support of a diagnostic tool for knee manipulation. In sum, embodiments of the invention may be used in many circumstances and clinical treatments that may require or suggest offloading for pain management and/or healing as well as for other reasons, conditions, and/or circumstances.

The advantages described herein are only examples of advantages and other advantages may be ascribed to the invention. In other words, the advantages herein are not an exhaustive listing. Moreover, one or more of the advantage examples may apply respectively to various differing embodiments of the invention.

Generally stated, the invention relates to a one-size-fits-all unloading and/or stabilizing knee brace. The knee brace may be used with any patient needing knee stabilization of either the left or right. Advantageously, the knee brace may be used from either the inside or outside of the left or right knee. The knee brace also may be used with a patient who is suffering pain from arthritis, bone on bone contact, and/or narrowed joint spacing in either left or right knee.

An exemplary embodiment of the invention may include a selectively activateable force device. It may exert pressure directly on the joint line of a patient's left or right knee and from either the inside or outside of the knee with which the knee is used when the force device is activated. The force device may exert pressure indirectly by pulling a knee strap wrapped around the left knee or the right knee of the patient and from either the inside or the outside of the left knee or the right knee with which the knee brace is used when the force device is activated. The result of the indirect pressure by the force device is that the knee strap applies the pressure to the joint line of the left knee or the right knee with which the knee brace is used.

The exemplary knee brace also includes a base positioned adjacent to the force device for at least partially resisting the pressure exerted by the force device towards the base. It also may support and/or protect the force device. The exemplary knee brace further includes a knee flexor positioned adjacent to the base for allowing the patient to at least partially flex the left knee or the right knee with which the knee brace is used when the knee brace is worn by the patient.

The knee flexor is connected to two extensions with each extension having two ends. One end of each extension is connected on respective top and bottom sides of the knee flexor so the two extensions extend away from the knee flexor with one of the extensions extending upwards along the left leg or the right leg of the patient with which the knee brace is used. The other of the connections extends downwards along the left leg or the right leg of the patient with which the knee brace is used. The other end of each extension is connected respectively to one of two cuffs.

One of the two cuffs is a top cuff for at least partially attaching the knee brace to the thigh corresponding to the left knee or the right knee of the patient with which the knee brace is used. The other of the two cuffs is a bottom cuff for at least partially attaching the knee brace to the calf corresponding to the left knee or the right knee of the patient with which the knee brace is used.

In addition, the exemplary knee brace includes attachment straps including a top attachment strap for attaching the knee brace to the thigh corresponding to the left knee or the right knee of the patient with which the knee brace is used. A bottom attachment strap attaches the knee brace to the calf corresponding to the left knee or the right knee of the patient with which the knee brace is used.

As noted, the exemplary knee brace includes a knee strap. It has an opening for positioning the knee strap around at least part of the patella of the left knee or the right knee of the patient with which the knee brace is used. The knee strap also includes dual connection straps on each of the left and right sides of the opening of the knee strap for wrapping around the back of the left knee or the right knee without hampering the popliteal crease and for securing the knee strap in position around the left knee or the right knee of the patient with which the knee brace is used.

Advantageously, the exemplary knee brace is one-size-fits-all.

The exemplary knee brace is usable on the outside of the left knee or the right knee of the patient without the knee strap to unload the medial compartment of the patient's left knee or right knee with which the knee brace is used by activation of the force device to exert pressure directly by pushing at the joint line on the outside of the left knee or the right knee with which the knee brace is used.

Also, the exemplary knee brace is usable on the inside of the left knee or the right knee without the knee strap to unload the lateral compartment of the patient's left knee or right knee with which the knee brace is used by activation of the force device to exert pressure directly by pushing at the joint line on the inside of the left knee or the right knee with which the knee brace is used.

Further, the exemplary the knee brace is usable on the outside of the left knee or the right knee of the patient with the knee strap to unload the lateral compartment of the patient's left knee or right knee by activation of the force device to exert pressure indirectly at the joint line on the inside of the left knee or the right knee with which the knee brace is used by pulling the knee strap wrapped around the patient's left knee or right knee with which the knee brace is used.

In addition, the exemplary knee brace is usable on the inside of the left knee or the right knee of the patient with the knee strap to unload the medial compartment of the patient's left knee or right knee by activation of the force device to exert pressure indirectly at the joint line on the outside of the left knee or the right knee with which the knee brace is used by pulling the knee strap wrapped around the patient's left knee or right knee with which the knee brace is used.

Additionally, the exemplary knee brace is usable on either the inside or the outside of the left knee or the right knee of the patient with or without the knee strap to provide for knee stabilization, to relieve pain from arthritis, to reduce bone on bone contact, or to compensate for decreased joint spacing in either the left knee or the right knee.

An embodiment of the knee brace invention may include a force device that is an air bladder that is selectively inflatable in increments. This embodiment may include a display on the knee brace for displaying one or more characters representing a respective increment or increments of inflation of the air bladder. This embodiment also may include a pump for inflating the air bladder. The pump may be integrally formed with the knee brace.

Other features and advantages of the invention may be more clearly understood and appreciated from a review of the following detailed description and by reference to the appended drawings and claims.

### BRIEF DESCRIPTION OF THE DRAWINGS

FIG. 1 shows the two knee joints of a person.

FIG. 2 shows an exemplary embodiment of the invention.

FIG. 3 shows an exemplary embodiment of the invention.

FIG. 4 shows an exemplary embodiment of the invention.

FIG. 5 shows an exemplary embodiment of the invention.

FIG. 6A shows an exemplary embodiment of the invention.

FIG. 6B shows a side view of some of the components of the exemplary embodiment shown in FIG. 6A.

FIG. 7 is a perspective view of an air bladder that may be used with an embodiment of the invention.

FIG. 8 shows a kit that may include components of an embodiment of the invention.

### DETAILED DESCRIPTION

The invention is described herein with reference to exemplary embodiments, alternative embodiments, and also with reference to the attached drawings. The invention, however, may be embodied in many different forms and carried out in a variety of ways, and should not be construed as limited to the embodiments set forth herein. The exemplary embodiments that are described and shown herein are only examples of the ways to implement the invention.

Herein, reference to a “knee brace” or “brace” also may include reference to one or more embodiments of a knee brace according to the invention, one or more embodiments of a kit for a knee brace according to the invention, one or methods of using same according to the invention, and/or one or more systems of using the knee brace and/or a kit for the knee brace as appropriate to the context and unless otherwise noted.

In terms of nomenclature herein, the singular includes the plural, the masculine includes the feminine or neutral, a person includes a corporation (and/or the like), and an embodiment, in whole or in part, may be an apparatus, a device, a kit, a method, and/or a system, unless otherwise noted. A user of an embodiment of the invention also may be referred to herein as a “patient.” A person who advises, aids, and/or instructs a patient on use of an embodiment of the invention may be referred to herein as a “clinician.” A “clinician” includes, but is not limited to a medical doctor, an orthotist, a medical sales representative, a pharmacist, and/or a retail sales clerk. Moreover, like numerals indicate like elements throughout the figures.

Generally stated, the exemplary knee brace may be used to unload the medial (inner) compartment of a knee or the lateral (outer) compartment of a knee. The exemplary knee brace may be used with either a patient's right knee or left knee. The exemplary knee brace may be used to unload the medial (inner) compartment of a knee or the lateral (outer) compartment of a knee applied or mounted to either the inside of the patient's afflicted knee or applied or mounted to the outside of the patient's afflicted knee.

For example, assume a first patient presents a need to have the medial (inner) compartment of his knee unloaded. The clinician may use the exemplary knee brace with the first patient. Working with the first patient, the clinician may choose to use the knee brace to unload the medial (inner) compartment of the patient's knee from either the inside or the outside of the patient's knee.

Assume a second patient presents a need for unloading, but of the lateral (outer) compartment of the patient's knee. The clinician may use the same exemplary knee brace for the second patient as the clinician used on the first patient. Working with the second patient, the clinician may choose to mount or apply the knee brace to unload the lateral (outer) compartment of the patient's knee from either the inside or the outside of the patient's knee.

Advantageously, the clinician does not have to find, purchase inventory, or place separate orders for a different knee brace for each type of unloading. The clinician may use an exemplary knee brace according to the invention for both types of unloading and for both types of manners of the particular type of unloading. Also, conveniently, the clinician does not have to find a different knee brace whether the clinician decides to cause the unloading from either the inside or the outside of the patient's knee, or for the patient's right or left knee.

More specifically, as noted, one of the two types of knee unloading that may be accomplished with the exemplary knee brace is that of unloading the medial (inner) compartment of the user's knee. The exemplary knee brace may be made to accomplish such unloading whether positioned on the outside or the inside of the user outside of the patient, in whole or in part, may be an apparatus, a device, a k unloading mechanism of the exemplary knee brace may be made to exert a “push” force from the outside of the user's knee and across it towards the inside of the knee to unload the medial (inner) compartment of the user's knee. When the knee brace is positioned on the inside of the user's knee, the force device and other elements of the exemplary knee brace may be made to exert a he across it from the outside of the user's knee and across it towards the inside of the knee to unload the medial (inner) compartment of the user's knee.

The other type of unloading is that of the lateral (outer) compartment of a patient's knee. The exemplary knee brace may be made to accomplish such unloading whether positioned on the outside or the inside of the user's knee. When the knee brace is positioned on the outside of the user's knee, the force device and other elements of the exemplary knee brace may be made to exert a “pull” force from the inside of the of the user's knee and across it towards the outside of the knee to unload the lateral (outer) compartment of the user's knee. When the knee brace is positioned on the inside of the user's knee, the force device of the exemplary knee brace may be made to exert a “push” force from the inside of the user's knee and across it towards the outside of the knee to unload the lateral (outer) compartment of the user's knee.

A clinician may have a different perspective than a patient in considering the advantages of the invention. The clinician may like the fact that he or she may apply an exemplary embodiment of the knee brace to the outside of Patient A's knee, and may apply the same embodiment of the knee brace to the inside of Patient Bps knee. In other words, the clinician may simply flip the same embodiment of the knee brace from the outside of Patient A's knee to the inside of Patient B's knee. Of course, the embodiment of the knee braces used on Patient A and Patient B is the same, but each patient has his or her own knee brace.

In sum, advantageously, the same exemplary embodiment of the invention may be used by a clinician with any patient's right or left knee to unload the patient's medial (inner) or lateral (outer) compartment of the afflicted knee from either the inside or the outside of the afflicted patient's knee. Moreover, the same exemplary embodiment may be put to use by a clinician in addition to or for other reasons than unloading as explained previously.

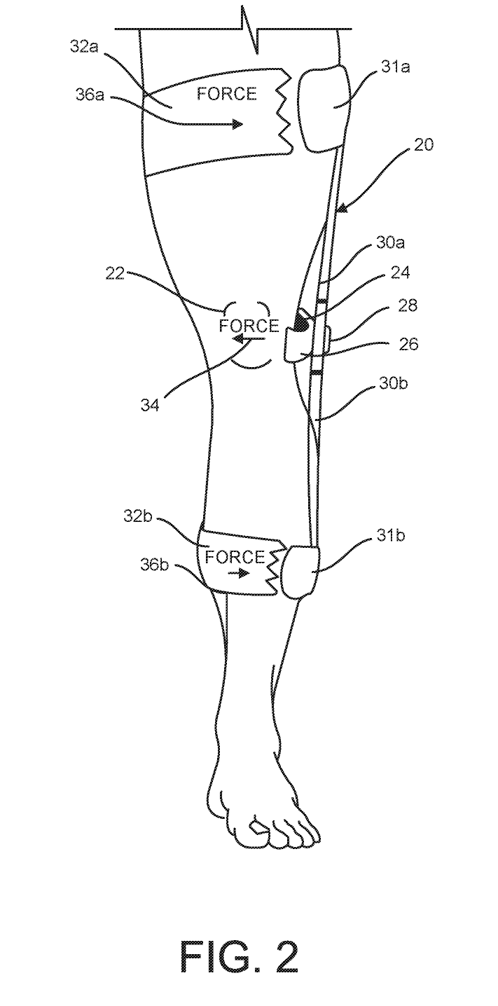

### FIG. 2—Exemplary Knee Brace 20 used for Unloading the Medial (Inner) Compartment of the User's Left Knee 22 from the Outside of the User's Knee 22

FIG. 2 illustrates the exemplary knee brace 20 as it may be used to unload the medial (inner) compartment of a patient's left knee 22. In particular, the knee brace 20 includes a force element 24 that may be positioned adjacent to the outside of the user's left knee 22. As used herein, the “outside” or “outer side” of a knee is the side of the knee furthest from the other leg, and the “inside” or “inner side” of a knee is the side of the knee closest to the other leg.

The force device 24 may be any mechanism or element that accomplishes the function described herein for force device 24. In the preferred embodiment, the force device 24 may be an air bladder. Other embodiments of a force device may include a foam or compressible ball, a screw mechanism, a spring mechanism, and/or a cam lever.

The force device 24 may be of any appropriate size and shape to accomplish its function. An embodiment may include a force device 24 that is about the size in height and width of the outside of an average person's knee. Other embodiments may vary.

When not activated, the force device 24 may be slightly concave on its side adjacent or nearest and with respect to the side of the patient's knee with which the knee brace is used. The slightly concave shape is preferably used to accommodate for the natural shape of the patient's knee on that side. In the exemplary embodiment 20, the force device 24 is shaped generally as an elongated “C.” The force device 24 is positioned on the outside of a user's knee so that its “C” shape points towards the user's knee 22. Other embodiments may vary.

When the force device 24 is activated, it may expand (but may retain a concave shape or “C” shape) so as to exert force or pressure in the direction intended. Preferably, the force device 24 may have a thickness (or depth) to accomplish the function of the force device 24 when it is activated. However, in the exemplary embodiment 20, the force device 24 may have a thickness still thin enough to maintain the “low profile” of the knee brace 20.

In the configuration of the exemplary knee brace 20 shown in FIG. 2, the force device 24 may be positioned adjacent to an element referred to herein as a “base” 26. In the exemplary embodiment, the base 26 may be substantially the same shape as the force device 24. Other embodiments may vary. Like the force device 24, the base 26 in the exemplary embodiment 20 may have an elongated “C” shape. In other embodiments, the force device 24 and the base 26 need not be the same general shape. In yet other embodiments, the force device 24 and/or the base 26 may be shaped other than the elongated nee brace ents throughout the base 26 may be slightly smaller or larger than the force device 24 at least in height and width (but not necessarily in thickness) to encompass the force device 24. For example, the base 26 may be thinner than the force device 24 at least when the force device is activated. Other embodiments may vary.

The base 26 may be constructed in any appropriate way to resist at least some of the force or pressure of the force device 24 when it is activated. By resisting at least some of the force of the activated force device 24, the base 26 may allow for more of the force to be directed as advantageous to the particular type of unloading of the knee.

In the exemplary embodiment 20, the force device 24 may be a separate element that may be positioned by the clinician and/or user next to the base 26 when the knee brace 20 is put together for its intended use with a particular patient. In an alternative embodiment, the force device 24 and base 26 may be made to be affixed to each other once they are positioned for a particular use. For example, if the alternative embodiment is a kit, then it may include an adhesive and/or other connector for the clinician and/or patient to use to connect the force device 24 and the base 26. In another embodiment, the force device 24 and base 26 may be integrally formed. In yet other embodiments, the force device 24 and base 26 may be provided to the clinician and/or user so that the force device 24 and base 26 are already positioned in the appropriate order depending on whether the knee brace is to be used for unloading the medial (inner) compartment or the lateral (outer) compartment of the user's knee and whether the knee brace is to be used with respect to the inside or outside of the patient's knee.

Reference now is made again to FIG. 2 and the respective positions of the elements of the exemplary knee brace 20 for unloading the medial (inner) compartment of the patient's knee 22 from its outside. On the side of the base 26 opposite to its position adjacent the force device 24, the base 26 is positioned adjacent to a hinge or lever 28. The hinge or lever 28 also may be referred to herein as the “knee flexor.” It may allow for the movement (such as bending) of the user's knee 22 while the knee brace 20 is being worn. Any hinge appropriate to the function may be used. The hinge 28 may include a range of motion control or not.

The hinge 28 in this example is generally rectangularly shaped, but does not necessarily have to be in all embodiments. The hinge 28 in this embodiment 20 may include or be connected to two extensions 30a, 30b that run substantially and respectively up and down the user's leg away from his or her knee 22. When a user is standing still, the extensions 30a, 30b may be aligned substantially vertically in line with respect to each other. The positions of the extensions 30a, 30b relative to each other and the hinge 28 may vary as the user walks, runs, sits, stands, and/or otherwise moves. Other embodiments may vary. Generally, the function of the extensions 30a, 30b is to connect the hinge 28 (thereby also connecting the force device 24 and base 26) with the attachments straps 32a, 32b and/or other elements such as 31a, 31b that hold the knee brace 20 to the patient's leg.

The extensions 30a, 30b may be made integrally with the hinge 28 in whole or in part, or may be separate elements from the hinge 28 and connected in any manner appropriate to function. The extensions 30a, 30b may be made of any material appropriate to their function. For example, the extensions 30a, 30b may be made of a substantially rigid material, such as metal, to facilitate a “lever” action with the hinge 28 for the knee brace 20. Both of the extensions 30a, 30b are made of the same material in embodiment 20, but that does not have to be the case in all embodiments. Further, the exemplary embodiment 20 includes two extensions 30a, 30b, but that does not necessarily have to be the case. Other embodiments may vary.

The lengths, widths, and thicknesses of the extensions 30a, 30b may be made sufficient to serve their respective connective purposes in the exemplary embodiment 20. In the exemplary embodiment 20, the respective lengths, widths, and thicknesses of the extensions 30a, 30b are substantially the same, but do not have to be the same in all or some features in other embodiments. The lengths of the extensions 30a, 30b are not readily adjustable in the exemplary embodiment 20, but other embodiments may include either extension 30a, 30b or both extensions 30a, 30b adjustable in length and/or in other ways. For example, one or both extensions in another embodiment may be made to be telescoping and/or otherwise adjustable.

In the exemplary embodiment 20, the extensions 30a, 30b are each about eight inches long, two inches wide, and have a thickness of about ⅛^{th} of an inch. Other embodiments may vary. For example, an embodiment of the invention may include extensions that are substantially or slightly contoured to correspond to the shape of a user's leg, that may be more ergonomic than other extensions serving the same function, and/or that may be more aesthetically pleasing.

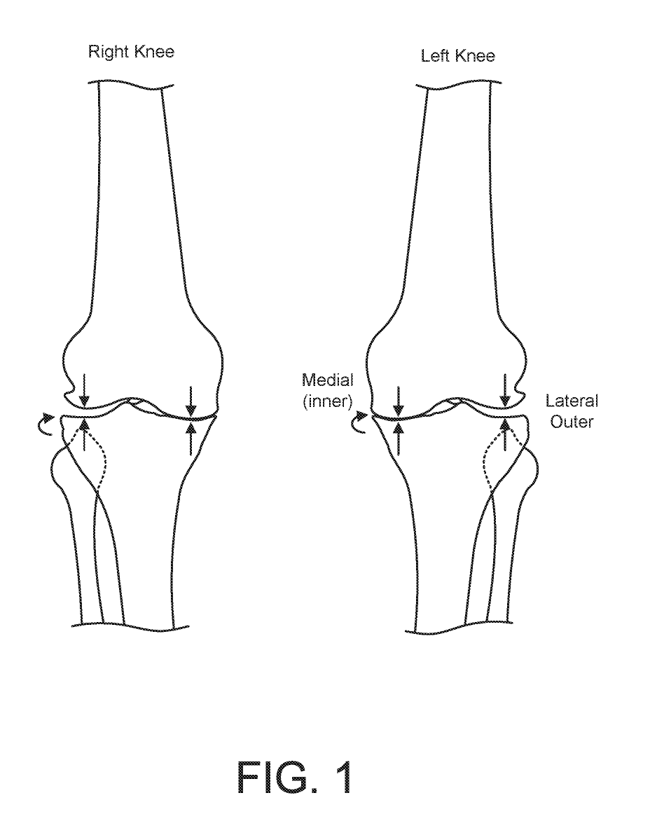

In the exemplary embodiment 20, the extensions 30a, 30b connect respectively at their ends opposite to their hinge connections to flanges 31a, 31b. Portions of the attachment straps 32a, 32b in FIG. 1 are shown in cutaway so the flanges 31a, 31b may be visible. A function of the flanges 31a, 31b, like the extensions 30a, 30b, in the exemplary embodiment, may be to connect the hinge 28, base 26, and force device 24 to the leg of the patient and to aid in keeping the knee brace 20 in proper position with respect to the user's knee 22. The flanges 31a, 31b also may be referred to as “cuffs.”

Each flange 31a, 31b generally is shaped as a “flattened” “C” so the “interior” of the “C” shaped flange may fit around at least part of the user's leg. Other embodiments may vary. In the exemplary knee brace 20, the extensions 30a, 30b connect respectively to the backs of their corresponding flanges 31a, 31b. The extensions 30a, 30b connect generally perpendicularly to their respective flanges 31a, 31b. More particularly, the long axes of the extensions 30a, 30b connect generally perpendicularly with respect to the long axes of the flanges 31a, 31b. The connections between the extensions 30a, 30b and the flanges 31a, 31b may be made in any appropriate manner to the function of the elements of the knee brace 20. In an alternate embodiment, the extensions 30a, 30b may be made integrally with the flanges 31a, 31b, but in this embodiment 20, they are separate elements.

Both flanges 31a, 31b are made of a material that may be at least somewhat flexible so that the flanges 31a, 31b may be made to conform to the contour of the user's leg. For example, the flanges 31a, 31b may be made of polypropylene, nylon, moldable metal, etc. Other embodiments may vary. Also, in this exemplary knee brace 20, the flanges 31a, 31b are both made of the same material, but that does not need to be the case in other embodiments.

The top flange 31a is about the same height along its short axis as the bottom flange 31b, but in this embodiment of the knee brace 20, the top flange 31a is about 6.5 inches long along its long axis in contrast to the 5.5-inch length of the bottom flange 31b. The difference in lengths of the top flange 31a and bottom flange 31b is to accommodate the generally larger girth of a patient's thigh over his or her calf, which are the areas of the leg where the flanges 31a, 31b are generally positioned. Of course, the flanges in other embodiments may vary. Alternative embodiments may allow for one or both of the flanges to be adjusted for a particular patient.

The exemplary knee brace 20 is held relatively and releasably fixed in position with respect to a user's leg with the aid of top and bottom straps 32a, 32b. They also may be referred to as “attachment straps.” In the exemplary embodiment 20, the attachment straps 32a, 32b are disposed substantially perpendicularly to the extensions 30a, 30b so the attachment straps 32a, 32b may be wrapped respectively around the flanges 31a, 31b and respectively the user's thigh and calf to generally releasably hold the knee brace 20 on the user's leg. Other embodiments may vary in connecting a knee brace according to the invention to a user's knee.

The attachment straps 32a, 32b may be made of material(s) and have features appropriate to their respective purposes in positioning and holding knee brace 20 adjacent to the outside of the user's knee 22 and in removing the knee brace 20 from the user's leg. The attachment straps 32a, 32b also may have a function in the transfer of force when the force device 24 is activated. Thus, the material and features of the attachment straps 32a, 32b may be made of material(s) that take into account this function regarding the transfer of force.

In an embodiment, the attachment straps 32a, 32b may be made of non-elastic webbing reinforced overlay. In another embodiment, each of the straps 32a, 32b may include an elastic base layer, which may promote compression and optimize the suspension of the knee brace 20 on the user's leg. In this embodiment, the base layer may be topped with a non-elastic strap (and/or other component), which may aid in or facilitate force transfer when the force device of the knee strap is activated.

Another embodiment of the invention may include straps 32a, 32b that are made of only semi elastic or elastic like material, which may aid in suspension of the knee brace on the user's leg and in migration (sliding and rotation) issues that may occur with respect to the knee brace for a patient. Other embodiments may include straps 32a, 32b of material in addition to or in place of those mentioned in this paragraph. Other embodiments may vary. For example, an embodiment may include attachment straps 32a, 32b made of a combination of elastic and non-elastic material. Yet another embodiment may include attachments straps 32a, 32b made of an elastic material. The attachments straps 32a, 32b may be both made of the same material, or they may differ with respect to each other.

In the exemplary embodiment 10, the attachment straps 32a, 32b may be about the same length, width, and thickness for each strap 32a, 32b. Alternative embodiments may include top and bottom straps that are different from each other in length, width, thickness, and/or other features. For example, the top attachment strap 32a may be wider and/or longer than the bottom attachment strap 32b in an embodiment of the invention. Yet other embodiments may include one or more additional straps to supplement either or both attachment straps 32a, 32b.

An embodiment of the invention may include attachments straps 32a, 32b as follows:
the top attachment strap 32a may include an elastic underlining that is about 28 inches long, four inches wide, and ⅛^{th} inch thick, and a non elastic overlay that is about 32 inches long, three inches wide, and 1/32 inch thick; and
the bottom attachment 32b may include an elastic underlining that is about 24 inches long, 3.5 inches wide, and ⅛^{th} inch thick, and a non elastic overlay that is about 28 inches long, 2.5 inches wide, and 1/32 inch thick.

An alternative embodiment of the invention may include attachment straps 32a, 32b (one or both) that may be trimmed to adjust to a patient's size. For example, the embodiment may provide attachment straps 32a, 32b that are longer than previously mentioned to accommodate a patient of larger than average size. This alternative embodiment may allow for the attachment straps 32a, 32b to be trimmed for a patient of a smaller size. The attachment straps 32a, 32b may be trimmed by the clinician and/or the patient if desired.

In the exemplary embodiment 20, the attachment straps 32a, 32b are fastened in the same manner with respect to each other, but the fastening mechanisms may be different between the upper attachment strap 32a and the lower attachment strap 32b in other embodiments. More particularly, the attachment straps 32a, 32b of the exemplary knee brace 20 are fastened via hook and loop closures such as the Velcro® brand. The closures may allow for adjustments of the respective fits of the attachment straps 32a, 32b to the user's particular thigh and/or calf dimensions. The hook and loop closures also may allow for re-adjustment of the attachment straps 32a, 32b as needed and/or desired. Other embodiments may vary.

Next is described an example of a method to attach the exemplary knee brace 20 to a patient's knee so the knee brace 20 may be made to unload the medial (inner) compartment of the patient's knee from the outside of the patient's knee 22. A clinician or a patient may carry out the actions described below in attaching the knee brace 20.

To attach the knee brace 20, the patient may be standing, sitting, or lying down, or otherwise positioned so the knee is not flexed or bent much. The knee brace 20 may be applied in other positions of the patient such as if his or her knee is bent, but such positioning of the patient may not be optimal. For unloading the patient patient's medial (inner) part of his or her left leg such as shown in FIG. 2, the force device 24 of the knee brace 20 may be positioned adjacent the outside (or outer side) of the patient's knee. If the base 26 is not already attached or positioned next to the outside of the force device 24, the base 26 may be so positioned next to the force device 24. If the hinge 28 is not already attached or positioned next to the outside of the base 26, then the hinge 28 may be so positioned next to the base 26. Next, the top extension 30a of the hinge 28 may be extended up away from the force device 24 as far as the top extension 30a may reach and/or as desired if the top extension 30a is adjustable. Once the top extension 30a is extended, the top flange 31a may be placed around at least a part of the patient's thigh. The attachment strap 32a then may be wrapped around the top flange 31a and the patient's thigh.

In a similar fashion, the bottom extension 30b of the hinge 28 may be extended down away from the hinge 28 as far as the bottom extension 30b may reach and/or as desired if the bottom extension 30b is adjustable. Once the bottom extension 30a is extended, the bottom flange 31b may be placed around the bottom flange 31b and the patient's calf. Some adjustments to the top attachment strap 32a and/or the bottom attachment strap 32b may be made as needed.

As an alternative to the above method, a clinician and/or user may choose to first position the bottom extension 30b, bottom flange 31b, and attachment strap 32b, and then position the top extension 30a, top flange 31b, and attachment strap 32a. Other methods of positioning an exemplary knee brace 20 are possible.

Once the knee brace 20 is positioned and relatively releasably secured on the patient's leg, its force device 24 may be activated so that it may exert a push force as indicated by arrow 34 across the patient's knee 22, thereby unloading the medial (inner) compartment of the patient's knee 22. The exertion of the push force across the patient's knee 22 may result in opposite or counter forces as indicated by arrows 36a, 36b respectively across the attachment straps 32a, 32b.

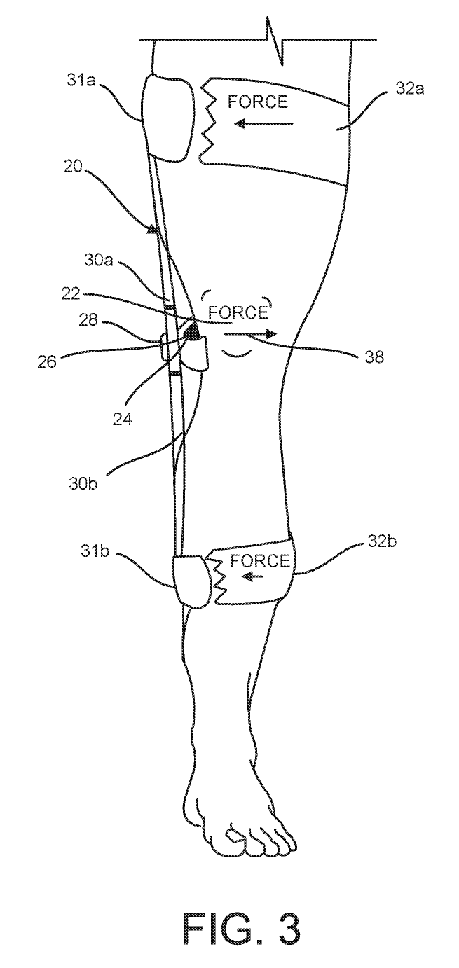

### FIG. 3—Exemplary Knee Brace 20 used for Unloading the Lateral (Outer) Compartment of the User's Left Knee from the Inside of the User's Knee 22

As noted above, the exemplary knee brace 20 may be used either to unload the medial (inner) compartment of a patient's knee or the lateral (outer) compartment of the patient's right or left knee and from either the inside or outside of the patient's knee 22. Advantageously, the same embodiment of the knee brace 20 described above in connection with FIG. 2 for unloading the medial (inner) compartment of a user's left knee from the outside of the user's knee 22 may be used to unload the lateral (outer) compartment of a patient's left knee from the inside of the patient's knee as shown in FIG. 3. Thus, a clinician may choose to use the same embodiment of a knee brace according to the invention whether a patient needs to unload his or her medial (inner) compartment or another patient needs to unload his or her lateral (outer) compartment, but the placement of the knee brace would be different (outside or inside the patient's knee respectively) depending on the type of unloading desired.

FIG. 3 is the same front view of the user's left leg 22 with the exemplary embodiment 20 as shown in FIG. 2 except that FIG. 3 shows the exemplary embodiment 20 on the inside of the patient's knee 22 in use for unloading the lateral (outer) compartment of the user's left knee 22.

In FIG. 3, the elements of the exemplary knee brace 20 are configured in the same order to unload the lateral (outer) compartment of the user's knee from the inside of the knee 22 in the same manner as the knee brace 20 was configured as shown in FIG. 2 to unload the medial (inner) compartment of the user's knee except of course that the knee brace 20 is positioned on the inside of the user's knee 22.

Referring to FIG. 3, the knee brace 20 includes the force device 24 positioned directly adjacent the inside of the user's knee 22. The base 26 is positioned adjacent to the force device 24 on its side opposite to the inside of the user's knee 22. When the base 26 is positioned outside of the force device 24, the base 26 may resist at least some of the force of the activated force device 24. By resisting at least some of the force of the activated force device 24, more of its force may be directed away from the base 26 and towards the knee 22 of the patient to contribute to unloading the lateral (outer) compartment of the user's left knee 22. Alternatively, and/or in addition, the base's resistance of the force from the force device 24 at least may reduce some of the force that otherwise may be dissipated in the direction away from the knee 22.

Like in the configuration of the exemplary knee brace 20 for unloading the medial (inner) compartment of the user configuration of the exemplary knee brace 20 on the inside of the user's knee for unloading the lateral (outer) compartment of the user's knee 22 g the la22 allows for the force device 24 to be simply positioned appropriately without affixation to the base 26. Alternatively, the force device 24 and base 26 may be affixed to each other as they are provided to the user and/or clinician in a kit and/or otherwise. As yet another alternative, the force device 24 and base 26 may be affixed to each other by the user and/or clinician. In even yet another embodiment, the force device 24 and base 26 may be integrally formed. Other embodiments may vary. As a further example, the force device 24, base 26, and hinge 28 may be integrated and pre-assembled for use with the knee brace. The pre-assembled force device 24, base 26, and hinge 28 may be included in a kit as described elsewhere herein.

Returning now to the high level description of the exemplary embodiment 20 shown in FIG. 3, on the side of the base 26 opposite to its position adjacent the force device 24, the base 26 may be positioned adjacent to the hinge or lever 28. It allows for the movement (such as bending) of the user's knee 22 while the knee brace 20 is being worn.

Whether used for either type of unloading, the knee brace's hinge 28 includes the top and bottom extensions 30a, 30b mentioned above in connection with FIG. 2.

Also for either type of unloading, the knee brace's top and bottom extensions 30a, 30b connect as previously described in connection with FIG. 2 respectively to top and bottom flanges 31a, 31b. Portions of the flanges 31a, 31b are shown in FIG. 3 in the cutaway areas of the attachment straps 32a, 32b. The attachment straps 32a, 32b may be wrapped respectively around the top and bottom flanges 31a, 31b and the user's thigh and calf to position and hold the knee brace 20 on the user's leg.

A method for positioning the exemplary embodiment 20 on the inside of the user's left knee 22 to unload the lateral (outer) compartment of the knee 22 compares to the method described above in connection with positioning the knee brace 20 on the outside of the user's left knee 22 to unload the medial (inner) compartment of the knee 22 except that the force device 24, base 26, hinge 28, extensions 30a, 30b, and top and bottom flanges 31a, 31b are positioned on the inside of the user's knee 22.

Once the knee brace 20 is positioned and relatively and releasably secured in place, its force device 24 may be activated so that it may exert a push force as indicated by arrow 38 across the patient's knee 22, thereby unloading the lateral (outer) compartment of the patient's knee 22. The exertion of the push force across the patient compartment of the patient's knee 22 may result in opposite or counter forces as indicated by arrows 40a, 40b respectively across the attachment straps 32a, 32b.

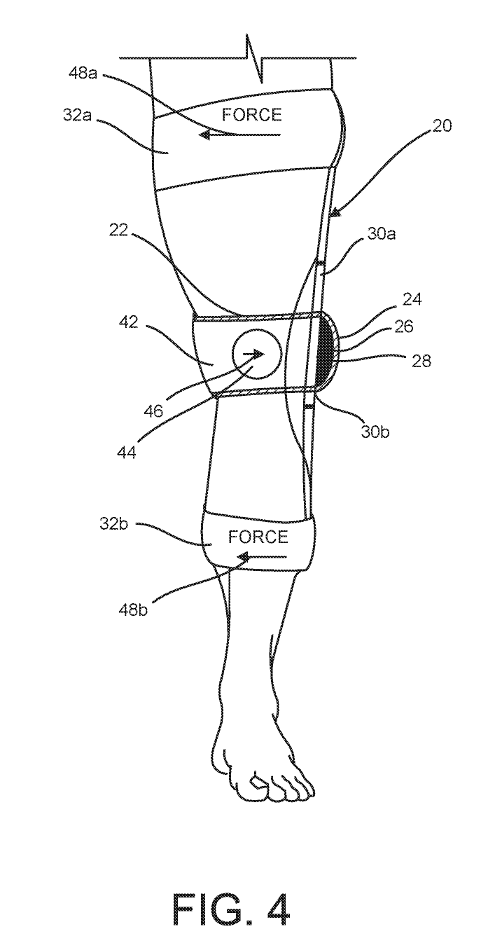

### FIG. 4—Exemplary Knee Brace 20 used for Unloading the Lateral (Outer Compartment) of the User's Left Knee 22 from the Outside of the User's Knee 22

As noted above, the exemplary knee brace 20 may be used either to unload the medial (inner) compartment of a patient's knee or the lateral (outer) compartment of the patient's right or left knee and from either the inside or the outside of the patient's knee 22. Advantageously, the same knee brace 20 described above in connection with FIG. 2 to unload the medial (inner) compartment of a user's left knee from the outside of the user's knee 22 also may be used from the outside of a patient's knee 22 to unload the lateral (outer) compartment of the patient's left knee as shown in FIG. 3.

However, in the configuration of the knee brace 20 shown in FIG. 4, the knee brace 20 is used with a knee strap 42 and a different configuration of some of the other elements of the knee brace 20 than the configuration shown above in FIG. 2. Generally stated, the function of the knee strap 42 and different configuration of some of the elements of the knee brace 20 with its placement on the outside of the patient's knee 22 in the embodiment of the invention illustrated in FIG. 4 is to exert a “pull” action on the user's knee joint that unloads the lateral (outer) compartment of the patient's knee when the force device 24 of the exemplary knee brace 22 is activated. Besides unloading the lateral (outer) compartment of a patient unloading the lateral (outer) compartment of a patient's knee, the knee brace 20 in the configuration shown in FIG. 4 also or alternatively may be used for other purposes (with or without unloading). As one example, without unloading, the knee brace 20 may be used in the configuration of FIG. 3 for a general instability application. As another example, the knee brace 20 may be used in the configuration of FIG. 4 for a condition(s) that may require range of motion control of the knee such as after surgery, and/or after a leg and/or knee injury.

FIG. 4 is the same front view of the user's left leg with the exemplary embodiment 20 on the outside of the patient's knee 22 in FIG. 2 except that FIG. 4 shows the exemplary embodiment 20 being used with a knee strap 42 and a different configuration of the elements of the knee brace 20 to unload the lateral (outer) compartment of the user's left knee 22. FIG. 4, unlike FIGS. 2 and 3, does not show the top and bottom flanges 31a, 31b which are hidden from view in FIG. 4 by the attachment straps 32a, 32b, which respectively cover the flanges 31a, 31b.

Referring to FIG. 4, the knee strap 42 is shown wrapped around the user's leg at the knee 22 with an opening 44 for the user's knee cap or patella. Part of the knee strap 42 on the outside of the user's knee 22 is shown in cutaway to allow a view of elements of the knee brace 20 lying below and covered by knee strap 42 when the knee brace 20 is in use.

In the embodiment 20 shown in FIG. 4, the hinge 28 is shown (by the cutaway of the knee strap 42) as positioned adjacent to the outside of the user's left knee 22. Other embodiments of the knee brace 20 may include one or more elements such as soft material between the hinge 28 and the outside of the user's left knee 22 to provide a cushion between the user's knee 22 and the hinge 28.

On the outside of the hinge 28, the base 26 is positioned. It may be positioned with respect to the hinge 28 so that the base 26 does not impede the functionality of the hinge 28.

Next to the outside of the base 26 is the force device 24. When positioned between the hinge 28 and the force device 24, the base 26 may resist at least some of the force of the activated force device 24. By resisting at least some of the force of the activated force device 24, more of its force may be directed away from the knee 22 and the hinge 28 and towards the knee strap 42 to add to the “pull force” that unloads the lateral (outer) compartment of the user's left knee 22. Alternatively, and/or in addition, the base's resistance of the force from the activated force device 24 at least may supplement some of the force that otherwise may be dissipated in the direction of the knee 22.

The hinge 28, base 26, and unloading mechanism 24 may be configured with respect to each other in terms of attachment and/or affixation and/or otherwise in any appropriate manner to carry out the function of this embodiment of the knee brace 20 shown in FIG. 4. See the discussions above associated with FIGS. 2 and 3 regarding the affixation, attachment, and/or positioning of these three elements 24, 26, 28 of the exemplary knee brace 20.

As noted, the knee strap 42 in embodiment 20 shown in FIG. 4 wraps around the user's leg and knee 22. The knee strap 42 also wraps around the knee brace 20, and in particular, wraps around the force device 24, which is positioned above the base 26, which, in turn, is positioned above the hinge 28.

When the force device 24 is activated, the force device 24 may cause a force to be directed towards the knee strap 42 that causes the knee strap 42 to distend in the direction of the force, which may be outwardly away from the knee 22. The distension of the knee strap 42 may cause a force as indicated by arrow 46 to be applied by the knee strap 42 from the inside of the user's left knee 22 across it and towards the outside of the user's knee 22, thereby unloading the lateral (outer) compartment of the user's knee 22. The force as indicated by arrow 46 may be considered a “pull” at least because the knee strap 42 may be considered to be “pulling” the user's knee so as to cause the lateral (outer) unloading. The exertion of the pull force across the patient arrow 46 may be considered “pulling” the user's knee so as to cause the lateral (outer) unloading. The exertion of the pull force across the patient's knee 22 may result in opposite or counter forces as indicated by arrows 48a, 48b respectively across the attachment straps 32a, 32b.

Next is described an example of a method to attach the exemplary knee brace 20 to a patient's knee so the knee brace 20 may be made to unload the medial (inner) compartment of the patient's knee from the outside of the patient's knee 22. A clinician or a patient may carry out the actions described below in attaching the knee brace 20.

To attach the knee brace 20, the patient may be standing, sitting, or lying down or otherwise positioned so the knee is not flexed or bent much. The knee brace 20 may be applied in other positions of the patient such as if his or her knee is bent, but such positioning of the patient may not be optimal. The hinge 28 with extensions 30a, 30b may be positioned adjacent to the outside (or outer side) of the patient's knee 22. The top and bottom flanges 31a, 31b may be positioned respectively around at least part of the user's thigh and calf. The attachment straps 32a, 32b then may be wrapped around the top and bottom flanges 31a, 31b and the user's thigh and calf. The attachment straps 32a, 32b may be re-adjusted later for better fit after the knee strap 42 has been wrapped around the user's knee 22 around and/or otherwise.

After the hinge 28 with extensions 30a, 30b and flanges 31a, 31b is in place on the outside of the user, 30b a 22, the base 26 may be positioned next to and on the outside of the hinge 28. The force device 24 then may be positioned next to and on the outside of the base 26. An alternative embodiment of the invention may provide the hinge 28 with base 26 and force device 24 already positioned and affixed as shown in FIG. 3 and ready to use in a kit as described elsewhere herein.

Once the base 26 and the force device 24 are in place on the hinge 28, the knee strap 42 may be applied to the patient's knee 22. The knee strap 42 may be applied by first placing the opening 44 in the knee strap 42 over the patient's knee. Then the remainder of the knee strap 42 may be wrapped around the patient's leg and knee 22 and the hinge 28 with connections 30a, 30b, base 26, and force device 24. The knee strap 42 may be releasably fixed to remain in its wrapped position in any appropriate manner.

Alternatively, the knee strap 42 may be applied to the user's knee prior to the wrapping of one or more attachment straps 32a, 32b around the patient's leg. Either or both the knee strap 42 and attachment straps 32a, 32b may be re-adjusted as desired or needed.

In the embodiment of the invention referenced in the previous paragraph, the knee strap 42 is described as a separate element. In an alternative embodiment, however, a knee strap having substantially the same function may be differently configured with respect to the other elements of that particular alternative embodiment. For example, a knee strap may be provided as already attached or releasably attached to one or more of the other elements in an embodiment of the invention prior to use of the embodiment by the clinician and/or patient. Such a knee strap, whether attached or releasably attached to the other elements of an embodiment, may be secured to the other elements using Velcro® or similar closing mechanisms, rivet(s), snaps, and/or other mechanisms. Advantageously, an embodiment of the invention having a knee strap already attached or releasably attached to the other elements of the embodiment prior to its use may provide convenience to the clinician and/or patient by at least saving the clinician and/or patient from having to make the attachment between the knee strap and the other element(s) of the alternative embodiment.

Once the knee brace 20 is positioned with the knee strap 42 wrapped in its relatively fixed but releasable position around the patient's knee 22, the force device 24 may be activated. As noted above, its activation may exert a force to distend the knee strap 42 away from the outside of the patient's knee 22. The distension of the knee strap 42 away from the outside of the patient's knee 22 may cause a pull force on the remainder of the knee strap 42 so that a force as indicated by arrow 46 is applied from the inside of the patient's knee across the patient's knee 22 towards the outside of the knee 22, thereby unloading the lateral (outer) compartment of the patient's knee 22. The exertion of the pull force 46 across the patient's knee 22 may result in opposite or counter forces as indicated by arrows 48a, 48b respectively across the attachment straps 32a, 32b.

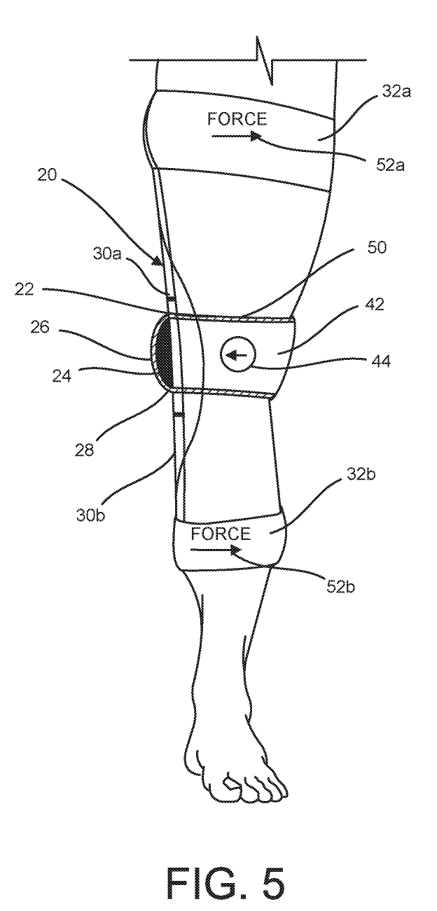

### FIG. 5—Exemplary Knee Brace 20 used for Unloading the Medial (Inner) Compartment of the User's Left Knee 22 from the Inside of the User's Knee 22

As noted above, the exemplary knee brace 20 may be used either to unload the medial (inner) compartment of a patient's knee or the lateral (outer) compartment of the patient's right or the patient's left knee and from either the inside or the outside of the patient's knee 22. Advantageously, the same knee brace 20 described above in connection with FIG. 3 to unload the lateral (outer) compartment of the user's left knee from the inside of the user's knee 22 also may be used from the inside of the user's knee 22 to unload the medial (inner) compartment of the patient's left knee as shown in FIG. 5.

However, in the configuration of the knee brace 20 shown in FIG. 5, the knee brace 20 is used with a knee strap 42 and a different configuration of some of the other elements of the knee brace 20 than the configuration shown above in FIG. 3. Generally stated, the function of the knee strap 42 and different configuration of some of the elements of the knee brace 20 with its placement on the inside of the patient's knee 22 in the embodiment of the invention illustrated in FIG. 5 may be to exert a “pull” action on the user's knee joint that unloads the medial (inner) compartment of the patient's knee 22 when the force device 24 of the exemplary knee brace 22 is activated. Nonetheless, the configuration of the knee brace 20 shown in FIG. 5 may be used on a patient's knee for additional or alternative reasons, force device 24 activated or not, such as for medial applications or in general instability applications.

FIG. 5 is the same front view of the user's left leg with the exemplary embodiment 20 on the inside of the patient's knee 22 as shown in FIG. 3 except that FIG. 5 shows the exemplary embodiment 20 being used with a knee strap 42 and a different configuration of the elements of the knee brace 20 to unload the medial (inner) compartment of the user's left knee 22. FIG. 5, like FIG. 4, but unlike FIGS. 2 and 3, does not show the top and bottom flanges 31a, 31b which are hidden from view in FIG. 5 by the attachment straps 32a, 32b, which respectively cover the flanges 31a, 31b.

Referring to FIG. 5, the knee strap 42 is shown wrapped around the user's knee 22 with an opening 44 for the user's knee cap or patella. Part of the knee strap 42 on the inside of the user's knee 22 is shown in cutaway to allow a view of elements of the knee brace 20 lying below and covered by knee strap 42 when the knee brace 20 is in use.

In the embodiment 20 shown in FIG. 5, the hinge 28 is shown (by the cutaway of the knee strap 42) as positioned adjacent to the inside of the user's left knee 22. Other embodiments of the knee brace 20 may include one or more elements such as soft material between the hinge 28 and the inside of the user's left knee 22 to provide a cushion between the user's knee 22 and the hinge 28.

On the side of the hinge 28 away from the knee 22, the base 26 is positioned. It may be positioned with respect to the hinge 28 so the base 26 does not impede the functionality of the hinge 28.

Next to the side of the base 26 away from the knee 22 is the force device 24. When positioned between the hinge 28 and the force device 24, the base 26 may resist at least some of the force of the activated force device 24. By resisting at least some of the force of the activated force device 24, more of its force may be directed away from the knee 22 and the hinge 28 and towards the knee strap 42 to add to the “pull” that unloads the medial (inner) compartment of the user's left knee 22. Alternatively, and/or in addition, the base's resistance of the force from the activated force device 24 at least may reduce some of the force that otherwise may be dissipated in the direction of the knee 22.

The hinge 28, base 26, and force device 24 may be configured with respect to each other in terms of attachment and/or affixation and/or otherwise in any appropriate manner to carry out the function of this embodiment of the knee brace 20 shown in FIG. 5. See the discussions above associated with FIGS. 2 through 4 regarding the affixation, attachment, and/or positioning of these three elements 24, 26, 28 of the exemplary knee brace 20.

As noted, the knee strap 42 in embodiment 20 shown in FIG. 5 wraps around the user's leg and knee 22. The knee strap 42 also wraps around the knee brace 20, and in particular, wraps around the force device 24, which is positioned to the outside of the base 26, which, in turn, is positioned outside of the hinge 28.

When the force device 24 is activated, the force device 24 may cause a force to be directed towards the knee strap 42 that causes the knee strap 42 to distend in the direction of the force, which may be away from the knee 22. The distension of the knee strap 42 may cause a force as indicated by arrow 50 to be applied by the knee strap 42 from the outside of the user's left knee 22 across it and towards the inside of the user's knee 22, thereby unloading the medial (inner) compartment of the user's knee 22. The force as indicated by arrow 50 may be considered a “pull” at least because the knee strap 42 may be considered to be “pulling” the user's knee so as to cause the medial (inner) unloading. The exertion of the pull force 50 across the patient's knee 22 may result in opposite or counter forces as indicated by arrows 52a, 52b respectively across the attachment straps 32a, 32b.

The method of attaching the exemplary knee brace 20 to a patient's knee 22 so the knee brace 20 may be made to unload the medial (inner) compartment of the knee 22 from the inside of the patient's knee 22 compares generally to the method described above in connection with FIG. 4 in attaching the exemplary knee brace 20 to a patient's knee 22 so the knee brace 20 may be made to unload the lateral (outer) compartment of the knee from the outside of the patient's knee with the exception that the hinge 28 with extensions 30a, 30b, flanges 31a, 31b, base 26, and unloading mechanism 24 are positioned on the inside of the patient's knee 22 as shown in FIG. 5. Thus, in the embodiment 20 shown in FIG. 5, the hinge 28 with extensions 30a, 30b and flanges 31a, 31b are in position on the inside of the user's leg and knee 22. The base 26 may be positioned next to and on the side of the hinge 28 away from the knee 22. The force device 24 then may be positioned next to and on the side of the base 26 away from the knee 22.

Once the hinge 28 with extensions 30a, 30b, base 26, flanges 31a, 31b, and unloading mechanism 24 are in place on the inside of the patient's knee, the knee strap 42 may be applied to the patient's knee 22. The knee strap 42 may be applied by first placing the opening 44 in the knee strap 42 over the patient's knee 22. Then, the remainder of the knee strap 42 may be wrapped around the patient's knee 22 and the hinge 28 with connections 30a, 30b, base 26, and unloading mechanism 26. In particular, the knee strap 42 may pass behind and encircle the patient's leg. The knee strap 42 may be releasably fixed to remain in its wrapped position in any appropriate manner. Alternatively, the knee strap 42 may be applied only after the attachment straps 32a, 32b are wrapped around the patient's leg. Additional details about the knee strap 42 and its use is described elsewhere herein.

In the embodiment of the invention referenced in the previous paragraph, the knee strap 42 is described as a separate element. In an alternative embodiment, however, a knee strap may be differently configured with respect to the other elements of that particular alternative embodiment. For example, a knee strap may be provided as already attached or releasably attached to one or more of the other elements in an embodiment of the invention prior to use of the embodiment by the clinician and/or patient. A knee strap, whether attached or releasably attached to the other elements of an embodiment, may be secured to the other elements using Velcro or similar closing mechanisms, rivet(s), snaps, and/or other mechanisms. Advantageously, an embodiment of the invention having a knee strap already attached or releasably attached to the other elements of the embodiment prior to its use may provide convenience to the clinician and/or patient by at least saving the clinician and/or patient from having to make the attachment between the knee strap and the other element(s) of the alternative embodiment.

Once the knee brace 20 is positioned with the knee strap 42 wrapped in its relatively releasably fixed position encircling the patient's leg at the knee 22, the force device 24 may be activated. As noted above, its activation may exert a force to distend the knee strap 42 away from the patient's knee 22. The distension of the knee strap 42 away from the patient's knee 22 may cause a pull force on the remainder of the knee strap 42 so that a force as indicated by arrow 50 may be applied from the outside of the patient's knee 22 across the patient's knee 22 towards the inside of the knee 22, thereby unloading the medial (inner) compartment of the patient's knee. The exertion of the pull force 50 across the patient's knee 22 may result in opposite or counter forces as indicated by arrows 52a, 52b respectively across the attachment straps 32a, 32b.

The knee strap 42 described above in connection with embodiments of the knee brace 20 illustrated in FIGS. 4 and 5 may be shaped like an “H.” In use, the knee strap 42 may be considered a “sideways” “H.” The upper arms of the “H” correspond to the left top and bottom strap elements of the knee strap 42; the lower arms (or legs) of the “H” correspond to the right top and bottom strap elements of the knee strap 42.

Instead of a cross bar like the conventional letter “H”, the knee strap 42 may include an opening 44 between the left top and bottom strap elements and the right top and bottom strap elements.

As noted, the knee strap 42 may be used in the embodiment 20 as if the “H” were in a sideways position. In that position, the opening 44 may fit over and around the user's knee cap. The left top and bottom strap elements of the “H” shaped knee strap 42 may be fastened to their respective right top and bottom strap element counterparts by wrapping the elements around and behind the patient's knee 22. Advantageously, the “arms” and “leg” of the “H” shaped knee strap 42 leave an opening behind the user's knee when they are unfastened. This opening behind the user's knee and between the fastened “arms and “legs” of the “H” shaped knee strap 42 allows the user to bend, and/or otherwise move or flex his or her leg at the knee. Particularly, the popliteal crease of the user's knee is not covered by the fastened knee strap 42 so that the user's use of his or her knee is not substantially hindered. The knee strap 42 may be fastened by hook and loop closures and/or otherwise. As noted previously, an alternate embodiment of the invention may include a knee strap that is provided already attached or releasably attached to other element(s) of the alternative embodiment.

More particularly, the exemplary knee strap 42 may include a generally circular opening 44. Other embodiments may have an opening of a different shape such as oval, square, or rectangular. The opening 44 may have a diameter corresponding to just slightly bigger than the diameter of an average person's knee cap. Other embodiments may have an opening 44 of a different shape and/or size. For example, an opening in a particular embodiment may be larger or smaller to fit a respectively larger or smaller knee cap.

The opening 44 in the knee strap 42 of the exemplary embodiment 20 may be made to fit over and around the patient's patella (knee cap). The opening 44 may leave the patella exposed so the knee brace 20 may accommodate the movement of the patient's knee when the knee brace 20 is worn. The size and/or shape of opening 44 may vary in other embodiments. The opening 44 may be of an adjustable size in an embodiment. In the exemplary embodiment 20, the opening 44 is integral with the remaining elements of the knee strap 42, but this may vary in other embodiments. For example, the opening 44 may be formed by a separate piece than the remaining elements of the knee strap 42.

The exemplary knee strap 42 may be positioned around the patient's knee and leg after the other elements of the knee brace 20 are in position on the patient's leg for unloading of the knee 22. To position the strap 42, the opening 44 may be placed over and around the patient's knee cap. The strap elements of the knee strap 42 may be wrapped around the patient's knee, around the other elements of knee brace 20, and/or around each other to fasten via hook and loop fasteners and/or otherwise. As noted previously, an alternate embodiment of the invention may include a knee strap that is provided already attached or releasably attached to other element(s) of the alternative embodiment. These may be positioned around the patient's knee and leg in the same general manner(s) described in connection with the knee strap 42, but taking into account their respective different characteristics.

Once the knee brace 20 is positioned for unloading of the patient's knee including the placement and fastening of the knee strap 42, the force device 24 may be activated as described above in connection with the embodiments shown in FIGS. 4 and 5.

Other alternative knee straps may be used in other embodiments. The material, size, shape, and other characteristics or features of the knee strap (in whatever embodiment) may be anything appropriate to the function of the knee strap with the invention.

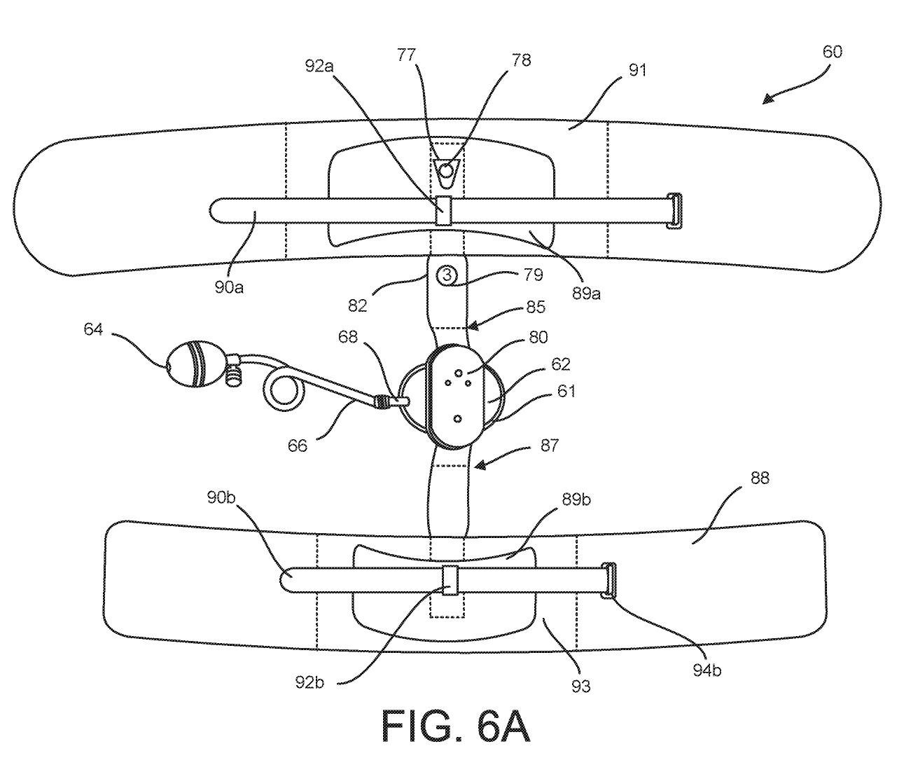

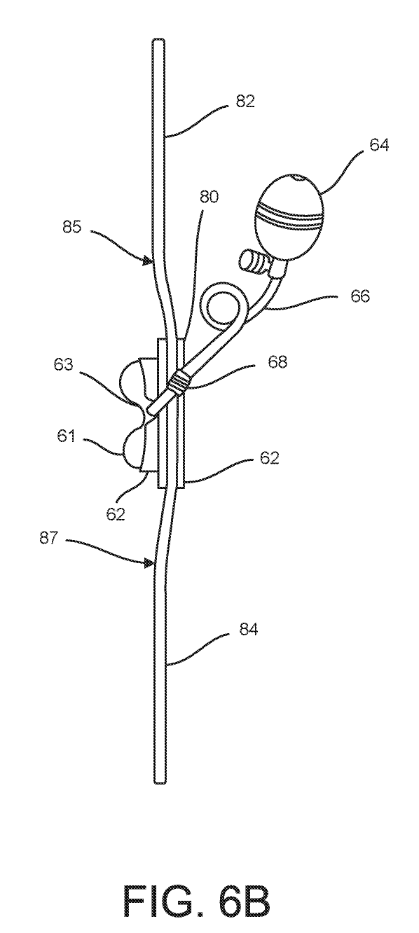

### FIGS. 6A, 6B, 7, and 8—Alternative Embodiments of the Invention

FIGS. 6A, 6B, 7, and 8 show alternative embodiments of the invention with reference to their respective components. In particular, FIGS. 6A and 6B show an alternative embodiment 60 of the invention or selected components with FIG. 6A showing the alternate embodiment 60 as its components may be put together in anticipation of use by a patient. FIG. 6B is a side profile view of selected components of alternative embodiment 60. These alternative embodiments may be used in the same manner as the embodiments previously described.

As explained, a patient may wear the invention such as embodied in knee brace 60 with a force device 61 adjacent to his or her left or right knee joint and on the inside or outside of his or her knee. FIG. 6A shows only part of force device 61 because it is substantially hidden by the base 62 in this view of the knee brace 60. FIG. 6B shows the force device 61 in profile. The location of the force device 61 behind the base 62 of the knee brace 60, even in FIG. 6A, is clear from the attachment of the air pump 64 via a tube 66 to a port 68 that is presumably on the force device 61 mostly hidden in FIG. 6A by the base 62, but visible in profile in FIG. 6B. The air pump 64 and tube 66 may be used with port 68 to inflate and/or deflate the force device 61 such as in the case where the force device 61 is an air bladder. The inflation of the force device 61 may be the “activation” of the force device, if it is an air bladder. As noted, a force device may be something other than an air bladder (in whole or in part) in other embodiments.

The force device 61 may be activated in increments in exemplary knee brace 60. The increments may vary from “0” activation to full or complete activation. The number of increments may vary from exemplary knee brace to knee brace, and/or from force device to force device used with a knee brace according to the invention. For example, if the force device 61 is an air bladder, it may be inflated a first amount (referred to as a “first increment”), which is less than full inflation, and it may be inflated a second amount (referred to as a “second increment”), which is more than the first increment, but less than full inflation, and so on.

An advantage of the activation of the force device 61 in increments may be to customize the force exerted by the force device 61 on the knee joint of the patient, and thus, to customize the exemplary knee brace 60 to the patient even though the exemplary knee brace 60 has universal application to patients. For example, a patient may use the exemplary knee brace 60 only as an aid to stabilize his or her left or right knee. In that case, the force device 61 may be left unactivated so the patient wears the exemplary knee brace 60 with the force device 61 adjacent his or her left or right knee, but with the force device 61 in its “least forceful” configuration in being unactivated. A force device that is unactivated in a knee brace may be referred to as having an increment of “0” activation. Also in that case, the force device 61 may serve as a cushion between the patient's knee and the other components of the exemplary knee brace 60.

As another example, a patient may activate the force device 61 to its third increment when wearing the exemplary knee brace 60. In this example, the force device 61 is made to exert a force on the joint line of the patient's left or right knee greater than if the force device 61 were activated to its first or second increment, but less than if the force device 61 were activated to its fourth increment (if the force device 61 has such an increment).

Advantageously, exemplary knee brace 60 provides for a display 79 of the activation increment on the knee brace 61 so that a patient may easily see what increment of the force device of the knee brace is being activated. The display 79 is integrally configured with exemplary knee brace 60, but other embodiments may vary. In FIG. 6A, the display 79 shows that the force device 61 is activated to its third increment by the character “3” shown on the display 79. Other embodiments may vary in the type and manner of display of the increments of activation (or no activation) of the force device of a knee brace according to the invention. The display 79 may be connected with the force device 61 and/or with the activator of the force device 61 to display the increment character on the display 79.

Reference now is made again to the air pump 64 and tube 66, which are shown in FIGS. 6A and 6B. The air pump 64 with tube 66 and port 68 are part of the mechanism in embodiment 60 that may accomplish the activation of the force device 61, which in this exemplary knee brace 60 is an air bladder. Other embodiments may have other activation mechanisms. The air pump 64 and tube 66 may be detachable. The port 68 also may be detachable in some embodiments. The knee brace 60 may include an integral pocket (not shown) and/or other component for storage of the air pump 64, tube 66, and/or port 68 and/or other element or accessory that may be used in connection with the exemplary knee brace 60.

FIG. 6A also shows an alternative activator 77 to the air pump 64, tube 66, and port 68 for use as an activation device for the force device 61 in the exemplary knee brace 60. The activator 77 may be integrally provided with the knee brace 60. In this example, the activator 77 is integrally provided as part of the top of the top extension 82 of the knee brace 60 shown in ghost form in FIG. 6A. Specifically, the activator 77 is integrally provided as part of the top of the top extension 82 that lies beneath or behind the thigh attachment strap 86. The positioning and construction of the activator 77 may be such that it is protected from damage when the exemplary knee brace 60 is worn and/or otherwise. The positioning and construction of the activator 77 also may be such that it is substantially hidden from view when the exemplary knee brace 60 is worn and/or otherwise. For example, the activator 77 may have an overall small size and shape, and/or have a profile that does not distend the thigh attachment strap 86 in a substantial manner.

In the exemplary knee brace 60, the activator 77 may include a button 78 or other device to activate the activator 77. The button 78 may be accessible to the patient from the outside of the attachment strap 86. As an example, the patient may push the button 78 once to cause the activator 77 to activate the force device 61 by one increment. Thus, if a patient desires to activate the force device 61 to its third increment, the patient may push the button 78 of the activator 77 three times. Other activators and/or buttons may vary. Advantageously, the activator 77 may be relatively small with respect to the knee brace 60 and may have a low profile such that its inclusion in the knee brace 60 and its use are convenient and comfortable for the patient.

The activator 77 may be an air pump if the force device 61 is an air bladder in embodiment 60. The activator 77 may be connected to a tube and/or other element(s) (not shown) that work with the activator 77 to activate the force device 61. An activator of another type than an air pump may include other and/or additional element(s) appropriate to its use.

In exemplary knee brace 60, the tube and/or other element(s) of the activator 77 may be positioned on the interior or inside of the top extension 82 from the activator 77 to the force device 61. Advantageously, the positioning of the tube and/or other element(s) on the inside of the top extension 82 may serve to protect the tube and/or other element(s) from damage when the exemplary knee brace 60 is in use by the patient and/or otherwise. Another advantage of positioning the tube and/or other element(s) on the inside of the top extension 82 is that the tube and/or other element(s) may be at least partially hidden from view by such positioning when the exemplary knee brace 60 is worn and/or otherwise.

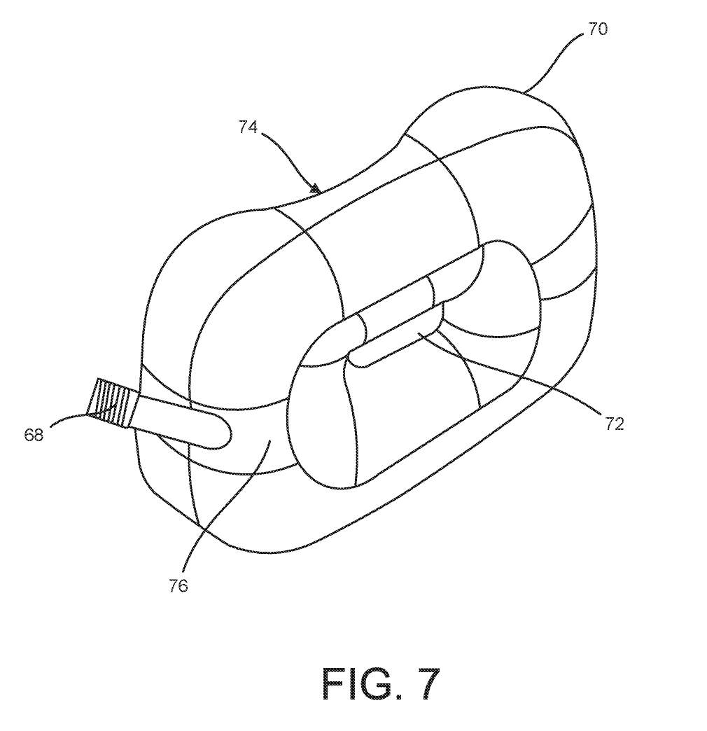

FIG. 7 shows an air bladder 70 that may be used as the selectively activateable force device of the exemplary knee brace 60. The air bladder 70 may be made selectively inflatable and deflatable. The air bladder 70 may be selectively inflatable or deflatable in increments. The air bladder 70 is shown with a port 68 (also shown in FIGS. 6A and 6B) for connecting to a device that may be used to inflate/deflate the air bladder 70. For example, an air pump 64 such as shown in FIGS. 6A and 6B may be used to inflate/deflate the air bladder 70. Other embodiments may vary in how the force device is activated.

The shape of the air bladder 70 may be characterized as a rectangular donut with rounded corners. The top and bottom sides of its generally rectangular shape are longer than its sides. In the embodiment of the air bladder 70 shown in FIG. 7, the top and bottom sides are each about 75 mm long. The front and back sides of the air bladder 70 are each about 50 mm in height. The air bladder 70 may vary in thickness (also referred to as “depth” or “side to side”) depending on whether the air bladder 70 is uninflated, partially inflated, or substantially fully inflated. The thickness of air bladder 70 in this embodiment may vary from about 10 mm to about 50 mm.

As noted, the air bladder 70 is characterized as having a “donut” shape because it has a centralized opening 72. As a “rectangular” donut, the air bladder 70 has an opening 72 that is also generally “rectangular.” Other embodiments may vary. An advantage of the opening 72 is that its inclusion maintains the shape of the air bladder 70 to be generally box-like (despite the rounded corners and walls) rather than becoming like a sphere or globe upon inflation. The opening 72 of the air bladder 70 may allow each of the side walls of the air bladder 70 to have a profile different from the curved profile that a sphere or a globe would have. The side walls 74, 76 of the air bladder 70 include the wall 74 of the air bladder 70 adjacent to the patient's knee (“adjacent wall 74”) and the wall 76 of the air bladder that is opposite to the adjacent wall 74. The profiles of the side walls 74, 76 of the “rectangular donut” shape of the air bladder 70 may be advantageous with respect to the pressure that may be exerted by the inflated air bladder 70 on the joint line of the knee on which the knee brace is worn. For example, rather than having a point or small area for applying pressure such as would happen if the air bladder 70 were spherical in shape, the “rectangular donut” shape of air bladder 70 may allow for more surface area in applying pressure. The air bladder 70 may be made of any appropriate material. The air bladder 70 may be activated by filling it with air (and/or other gas and/or substance).

The air bladder 70 is characterized above as having a rectangular donut shape with rounded corners. The side walls 74, 76 of the air bladder 70 are those sides that lie between the top and bottom sides and the sides. As noted, one of the side walls 74 is positioned directly adjacent a patient's knee and may be in touch with the skin of the person's knee. This side wall 74 is referred to herein as the adjacent side wall 74. The other side wall 76 of the air bladder 70 is opposite to the adjacent side wall. The other side wall 76 is referred to herein as the outer side wall 76.

An advantage presented by air bladder 70 is that its adjacent side wall 74 may be at least slightly concave or “C” shaped. The concave shape of the adjacent side wall 74 is from the front to the back of the patient's knee. The concave shape of the adjacent side wall 74 may more readily conform to the shape of a patient's knee than another shape. The adjacent side wall 74 may have a concave shape uninflated, or when partially or fully inflated. As noted, an air bladder may be inflated in “increments.” The adjacent side wall 74 of such an air bladder may retain its concave or “C” shape when it is inflated increment by increment. FIG. 6B presents a profile view of force device 61, which compares to the air bladder 70 of FIG. 7. The generally concave shape of the adjacent side wall of force device 61 is indicated by the curved line 63. Advantageously, a concave shape of the adjacent side wall of a force device used in an exemplary embodiment of the invention may conform better than other shapes with respect to the shape of the knee of a patient with which the knee brace is used.

Referring again to embodiment 60 shown in FIGS. 6A, 6B, it has been noted that the base 62 substantially hides the force device 61 of that embodiment 60 as shown in FIG. 6A. The base 62 may be positioned generally adjacent to the force device 61 to support it. In embodiment 60, the base 62 is shaped to more or less match the height and width of the outer side wall 76 of the force device 61. Thus, the base 62 may be shaped as a rounded rectangle with a bit of depth. Another point is that base 62 may be said to substantially completely support force device 61 by being of about the same size as the force device 61. This may be an advantage in that the base 62 may protect the force device 61 when the knee brace 60 is in use and/or otherwise. Other embodiments may vary in respective sizes between a force device and a base. The base 62 may be connected to the force device 61. If there is such a connection, it may be made in any appropriate manner. Alternative embodiments of the invention may omit a base.

Besides supporting the force device, the base 62 of embodiment 60 may also serve the function of at least partially resisting the pressure exerted by the force device 61 towards the base 62 such as when the force device 61 is activated. For example, assume an air bladder is the force device 61 in embodiment 60. Upon inflation, the air bladder expands towards the base 62 as well as in other directions. The pressure of the expansion of the air bladder is at least partially resisted by the base 62 so that the overall configuration of the knee brace 60 is not distended as would otherwise be distended outwards from the knee with which the knee brace is used, and so that the pressure may, instead of being applied towards the base 62, be directed otherwise such as in the direction of the joint line of the knee to which the knee brace is attached.

On the side of the base 62 opposite to its attachment to the force device, the base 62 may be connected to a knee flexor 80. The connection may be made in any appropriate manner. The knee flexor also may be referred to herein as a “hinge.” The knee flexor 80 may move clockwise and counter-clockwise manner. Other embodiments may vary. The knee flexor 80 may allow the patient to at least partially flex the left knee or the right knee with which the knee brace is used when the exemplary knee brace 60 is worn by the patient. The exemplary knee flexor 80 may include range of motion control features (not shown in the figures). An alternative embodiment of the invention may omit range of motion control features. In the exemplary embodiment 60, the knee flexor 80 is about 40 mm wide and about 75 mm in length.

In embodiment 60, the knee flexor 80 is connected to two extensions 82, 84 that run substantially and respectively up and down the patient's leg away from his or her knee. When a patient is standing still, the extensions 82, 84 may be aligned substantially vertically in line with respect to each other. The positions of the extensions 82, 84 relative to each other and the knee flexor 80 may vary as the user walks, runs, sits, stands, and/or otherwise moves. Other embodiments may vary. Generally, the functions of the extensions 82, 84 may include connecting the knee flexor 80 (thereby also connecting the force device 61 and base 62) respectively with the attachments straps 86, 88 that hold the exemplary knee brace 60 to the patient's leg.

The extensions 82, 84 may be made integrally with the knee flexor 80 in whole or in part, or may be separate elements from the knee flexor 80 and connected in any manner appropriate to function. The extensions 82, 84 may be made of any material appropriate to their function. For example, the extensions 82, 84 may be made of a substantially rigid material, such as metal, to facilitate a “lever” action with the knee flexor 80 for the knee brace 60. Both of the extensions 82, 84 are made of the same material in embodiment 60, but that does not have to be the case in all embodiments. Further, the exemplary embodiment 20 includes two extensions 82, 84, but that does not necessarily have to be the case. Other embodiments may vary.

The lengths, widths, and thicknesses of the extensions 82, 84 may be made sufficient to serve their respective connective purposes in the exemplary embodiment 60. In the exemplary embodiment 60, the respective lengths, widths, and thicknesses of the extensions 82, 84 are substantially the same, but do not have to be the same in all or some features in other embodiments. The lengths of the extensions 82, 84 are not readily adjustable in the exemplary embodiment 60, but other embodiments may include either extension 82, 84 or both extensions 82, 84 adjustable in length and/or in other ways. For example, one or both extensions in another embodiment may be made to be telescoping and/or otherwise adjustable.

In the exemplary embodiment 60, the extensions 82, 84 are each about 193 mm long and 30 mm wide. Other embodiments may vary. For example, an embodiment of the invention may include extensions that are substantially or slightly contoured to correspond to the shape of a patient's leg, that may be more ergonomic than other extensions serving the same function, and/or that may be more aesthetically pleasing.

FIGS. 6A and 6B include bend lines 85, 87 respectively in the top and bottom extensions 82, 84. Each bend line 85, 87 may traverse the width of its respective extension 82, 84. Each bend line 85, 87 also may be positioned a distance of one-third along the length of the extension 82, 84 from about the center of the knee flexor 80. Each bend line 85, 87 may allow its respective extension 82, 84, and thus, the knee brace 60, to be configured to the contours of a particular patient's leg. For example, one of the extensions may be bent towards or away from the patient's leg along the bend line of the extension to customize the fit of the knee brace to the patient. Embodiment 60 includes a bend line 85, 87 in each extension 82, 84, but other embodiments may include fewer or more bend lines. Other embodiments may provide other or additional ways in which the extensions 82, 84 may be configured to conform to the contours of a particular patient's leg. The bend lines 85, 87 and/or the functionality of being able to adjust the configurations of the extensions 82, 84 of the exemplary knee brace 60 are an advantage in providing a knee brace that may have near universal application yet be specifically configured for a unique individual.

In the exemplary embodiment 60, the extensions 82, 84 may connect respectively at their ends opposite to their hinge connections to flanges 89a, 89b shown in ghost form as respectively lying underneath attachment straps 86, 88. A function of the flanges 89a, 89b, like the extensions 82, 84, in the exemplary embodiment 60, may be to connect the hinge 80, base 62, and force device 61 to the leg of the patient and to aid in keeping the knee brace 60 in substantially proper position with respect to the patient's knee. The flanges 89a, 89b also may be referred to as “cuffs.”

Each flange 89a, 89b may be generally shaped as a rectangle with rounded corners that is shaped to resemble a “flattened” “C.” The “interior” of the “C” shaped flange may fit around at least part of the patient's leg. Other embodiments may vary. In the exemplary knee brace 60, the extensions 82, 84 may connect respectively to their corresponding flanges 89a, 89b. The extensions 82, 84 may connect generally perpendicularly to their respective flanges 89a, 89b. More particularly, the long axes of the extensions 82, 84 may connect generally perpendicularly with respect to the long axes of the flanges 89a, 89b. The connections between the extensions 82, 84 and the flanges 89a, 89b may be made in any appropriate manner to the function of the elements of the knee brace 60. In an alternate embodiment, the extensions 82, 86 may be made integrally with the flanges 89a, 89b, but in this embodiment 60, they are separate elements.

Both flanges 89a, 89b are made of a material that may be at least somewhat flexible so that the flanges 89a, 89b may be made to conform to the contour of the patient's leg. For example, the flanges 89a, 89b may be made of polypropylene, nylon, moldable metal, etc. Other embodiments may vary. In this embodiment, the flanges 89a, 89b lend some rigidity to the upper and bottom elements of the exemplary knee brace 60. Also, in this exemplary knee brace 60, the flanges 89a, 89b are both made of the same material, but that does not need to be the case in other embodiments.

In embodiment 60, the top flange 89a is about the 101 mm along its short axis and about 255 mm along its long axis. The bottom flange 89b is about 76 mm along its short axis and about 203 mm along its long axis. The difference in sizes between the flanges 89a, 89b may be to accommodate the generally larger girth of a patient's thigh over his or her calf, which are the areas of the leg where the flanges 89a, 89b are generally positioned. Of course, the flanges in other embodiments may vary. Alternative embodiments may allow for one or both of the flanges to be adjusted in other ways than mentioned herein for a particular patient.

Advantageously, in the exemplary embodiment 60, each of the flanges 89a, 89b with its respectively connected extension 82, 84 may be made to slip respectively into a pocket 91, 93 created as part of an attachment strap 86, 88. The pockets 91, 93 may be sized and shaped in a manner to accept respectively the flanges 89a, 89b and at least part of the connected extensions 82, 84. The part of each pocket 91, 93 of the attachment straps 86, 88 adjacent a patient's leg may provide cushioning for the patient with respect to the flanges 89a, 89b and connected extensions 82, 84. Additional cushioning may be provided in other embodiments. Once the flanges 89a, 89b with their connected extensions 82, 84 are in place in the pockets 91, 93 of the attachment straps 86, 88, the pockets 91, 93 may be secured so the flanges 89a, 89b with their connected extensions 82, 84 are reasonably secured within the pockets 91, 93. Securing the pockets 91, 93 may allow for at least some continued movement of the flanges 89a, 89b with their connected extensions 82, 84 as appropriate to the function of the knee brace 60.

An advantage of the use of pockets 91, 93 in attachment straps 86, 88 is that one or both of the attachment straps 86, 88 may be replaced if one and/or the other becomes damages, wears out, and/or for any other reason. Thus, a patient does not have to invest in a new knee brace according to the invention if one or both of the attachment straps is not usable, but instead, may replace just the attachment strap(s) rather than the whole knee brace.

The attachment straps 86, 88 hold the exemplary knee brace 60 relatively and releasably relatively fixed in position at least in part with respect to a patient's leg. In the exemplary embodiment 60, the attachment straps 86, 88 are disposed substantially perpendicularly to the extensions 82, 84 so the pockets 91, 93 of the attachment straps 86, 88 may be accept respectively the flanges 89a, 89b and at least part of the connected extensions 82, 84. The attachment straps 86, 86 are wrapped respectively around the user's thigh and calf to generally releasably hold the knee brace 60 on the patient's leg.

The attachment straps 86, 88 may be made secure in any appropriate manner. In the exemplary embodiment 60, the ends of each attachment strap 86, 88 may be made to secure to each other by the use of a piece of connecting fabric such as Velcro®. Each end of an attachment strap 86, 88 may be provided with either a hook or loop portion of a Velcro® type connector, and a piece of material with the other type (loop or hook) portion may be positioned so that the ends of an attachment strap 86, 88 are connected to each other and/or to other part(s) of the same attachment strap 86, 88. Other embodiments may.

The attachment straps 86, 88 may be made of material(s) and have features appropriate to their respective purposes in positioning and holding knee brace 60 adjacent to the outside of the patient's knee and in removing the knee brace 60 from the user's leg. The attachment straps 86, 88 also may have a function in the transfer of force when the force device 61 is activated. Thus, the material and features of the attachment straps 86, 88 may be made of material(s) that take into account this function regarding the transfer of force.

In an embodiment, the attachment straps 86, 88 may be made of non-elastic webbing reinforced overlay. In another embodiment, each of the straps 86, 88 may include an elastic base layer, which may promote compression and optimize the suspension of the knee brace 60 on the patient's leg. In this embodiment, the base layer may be topped with a non-elastic strap (and/or other component), which may aid in or facilitate force transfer when the force device of the knee strap is activated.

Another embodiment of the invention may include straps 86, 88 that are made of only semi elastic or elastic like material, which may aid in suspension of the knee brace on the user's leg and in migration (sliding and rotation) issues that may occur with respect to the knee brace for a patient. Other embodiments may include straps 86, 88 of material in addition to or in place of those mentioned in this paragraph. Other embodiments may vary. For example, an embodiment may include attachment straps 86, 88 made of a combination of elastic and non-elastic material. Yet another embodiment may include attachments straps 86, 88 made of an elastic material. The attachments straps 86, 88 may be both made of the same material, or they may differ with respect to each other.

In the exemplary embodiment 60, the attachment straps 86, 88 are generally rectangular with rounded corners. The attachment straps 86, 88 in this embodiment 60 vary in size from each other. Other embodiments may vary. The top attachment strap 86 may be about 712 mm along its long axis and about 150 mm along its short axis. The bottom attachment strap 88 is about 560 mm along its long axis and about 110 mm along its short axis. In an alternative embodiment, the attachment straps may taper in width near their respective ends. The tapering may minimize the amount of material that may have to be wrapped around a patient's knee. The tapering is another advantage that allows embodiments of the invention to be near universal in application to patients of varying sizes and shapes, but yet customized to a particular person.

Exemplary embodiment 60 includes additional straps 90a, 90b to aid in positioning and releasably securing the knee brace 60 to a patient's leg. These additional straps may be referred to as securement straps 90a, 90b. One of each is used respectively with attachment straps 86, 88. Other embodiments may use fewer or more such straps and/or alternative straps. Each securement strap 90a, 90b is generally rectangular in shape, but much longer than wide. The top securement strap 90a is about 275 mm in length, but only about 50 mm in width. The bottom securement strap 90b is about 220 mm in length and about 50 mm in width.

Generally stated, once the attachments straps 86, 88 are positioned around a patient's leg, then the securement straps 90a, 90b may be positioned and releasably fastened around the respective attachment straps 86, 88. Whether the top securement strap 90a or the bottom securement strap 88 is positioned and/or fastened first or second with respect to the other securement strap is up to the patient and/or the clinician. The same may be said with respect to the attachment straps 86, 88. Moreover, the securement straps 90a, 90b may take several rounds of positioning and fastening to reach an optimal or most comfortable fit. Once a patient is familiar with the use of the exemplary knee brace 60, the patient may learn quickly which position and fastening of the securement straps 90a, 90b are best for him or her. The same may be said with respect to the attachment straps 86, 88.

Also generally stated, the securement straps 90a, 90b in the exemplary knee brace 60 are positioned generally around the lower part of the top attachment strap 86 and around the upper part of the bottom attachment strap 88. There may be some advantages to such relative placement of the securement straps 90a, 90b. One of the advantages is that such placement of the securement straps 90a, 90b aids in preventing or minimizing bow out or bulging out of other components of the exemplary knee brace 60 when the force device 61 is activated. In other words, the securement straps 90a, 90b may aid in keeping components of the exemplary knee brace 60 in proper position with respect to each other so as to maximize the benefit of the knee brace 60 to the patient.

FIG. 6A shows that the securement straps 90a, 90b may be attached respectively to attachment straps 86, 88 by the securement straps 90a, 90b being threaded through a loop 92a, 92b. Each securement strap 90a, 90b may be threaded through its respective loop 92a, 92b in a similar manner to which a belt is threaded through a belt loop. The ends of each securement strap 90a, 90b may be secured with respect to each other to secure the securement straps in their respective positions around the attachment straps 86, 88. In the exemplary embodiment 60, one end of each securement strap 90a, 90b is provided with a metal loop 94a, 94b. The other end of each securement strap 90a, 90b (referred to herein as the “threaded end”) is provided along part of its outer side with one element of a hook and eye closure system (such as Velcro®). The threaded end may be pushed through the metal loop and folded back on its securement strap to mate with the other element of the hook and eye closure system disposed along the outer side of the remainder of the securement strap. In other words, the securement strap may be folded back on itself and secured after it passes through its metal loop. Advantageously, each of the securement straps 90a, 90b may be releasably secured in an adjustable fit customized to the patient.

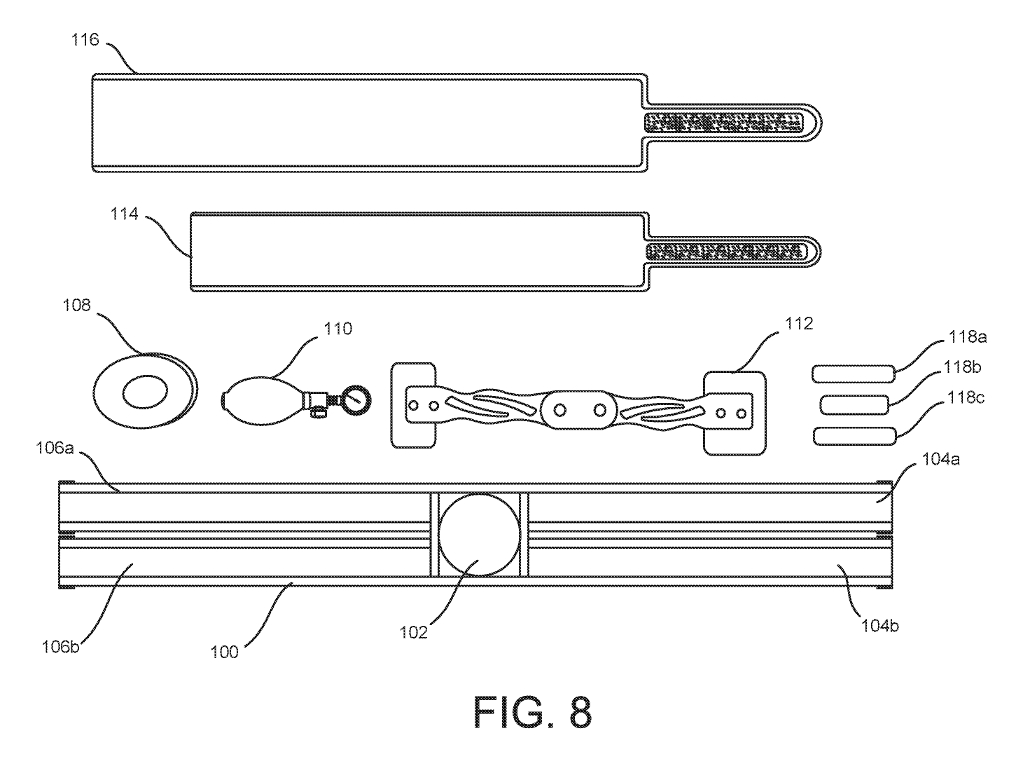

FIG. 8 shows a knee strap 100 as may be used with embodiment 60. The knee strap 100 is shown as it may be included (as discussed below) with other knee brace components in a kit 99 for an exemplary knee brace.

Overall, knee strap 100 is about 20 inches long and about 5 inches wide. Other embodiments may vary. Knee strap 100 includes a central generally square area having an opening 102, which is circular and about 3 inches in diameter. The opening 102 may be placed over the patella (knee cap) of a patient's knee when the knee strap 100 is used with the exemplary embodiment 60 (and/or other embodiments).

The knee strap 100 includes a set of dual connection straps 104a, 104b, 106a, 106b on each of the left and right sides of the opening of the knee strap 100. Specifically, the right side of knee strap 100 as shown in FIG. 8 includes two straps 104a, 104b positioned one 104a above the other 104b with respect to the central area of the knee strap 100. The left side of the knee strap 100 as shown in FIG. 8 includes two straps 106a, 106b positioned one 106a above the other 106b with respect to the central area of the knee strap 100. The dual connection straps 104a, 104b, 106a, 106b may be made to wrap the knee strap 100 around the back of the left knee or the right knee of the patient. Advantageously, the dual connection straps wrap the knee strap 100 around the patient's knee without hampering the popliteal crease of the left knee or the right knee of the patient with which the knee brace is used. The dual connection straps 104a, 104b, 106a, 106b may be connected or secured in place in any appropriate manner. The exemplary knee strap may use hook and eye (Velco™) closures. Particularly, the respective tops 104a, 106a of the dual connection straps connect to each other and the respective bottoms 104b, 106b connect to each in a manner to allow the patient to flex his or her knee at least somewhat.

Besides the knee strap 100 that may be part of the kit 99 shown in FIG. 8, other elements of the kit 99 may include an adjustable air bladder 108 to function as a force device. An inflation bulb 110 is included as the activator to inflate the air bladder 108. Component 112 includes a knee flexor identified as including bidirectional hinges with range of motion (ROM). Component 112 also includes top and bottom extensions as well as top and bottom flanges or cuffs. A calf wrap 114 and a thigh wrap 116 also are included in the kit 99. Conveniently, the kit 99 includes three Velcro® attachments tabs 118a, 118b, 118c of various sizes that may be used with the components of the kit 99. Advantageously, the kit 99 provides an exemplary knee brace according to the invention that is of near universal application for most individuals, that may be used on either a left or a right knee of a patient, that may be used on the inside or outside of a knee of a patient, but that yet may be customized for a particular patient to optimize the use of the exemplary knee brace for that person.

### CONCLUSION

The exemplary embodiments of the invention were chosen and described above in order to explain the principles of the invention and its practical applications so as to enable others skilled in the art to utilize the inventions including various embodiments and various modifications as are suited to the particular uses contemplated. The examples provided herein are not intended as limitations of the present invention. Uses with other types of apparatus, devices, items, methods, and/or systems may be appropriate. Other embodiments will suggest themselves to those skilled in the art.

1. A one-size-fits-all unloading and/or stabilizing knee brace for any patient needing knee stabilization of either the left or right knee and/or from either the inside or outside of the left or right knee, and/or suffering pain from arthritis, bone on bone contact, and/or narrowed joint spacing in either left or right knee, comprising:
a selectively activateable force device for exerting pressure directly by pushing at a joint line of either a left knee or a right knee of a patient and from either the inside or the outside of the left knee or the right knee with which the knee brace is used when the force device is activated, or
the selectively activateable force device being for exerting the pressure indirectly by pulling a knee strap wrapped around the left knee or the right knee of the patient and from either the inside or the outside of the left knee or the right knee with which the knee brace is used when the force device is activated that results in the knee strap applying the pressure to the joint line of the left knee or the right knee with which the knee brace is used;
a base positioned adjacent to the force device for at least partially resisting the pressure exerted by the force device towards the base;
a knee flexor positioned adjacent to the base for allowing the patient to at least partially flex the left knee or the right knee with which the knee brace is used when the knee brace is worn by the patient;
two extensions with each extension having two ends,
one end of each extension being connected on respective top and bottom sides of the knee flexor so the two extensions extend away from the knee flexor with one of the extensions extending upwards along the left leg or the right leg of the patient with which the knee brace is used and the other of the connections extending downwards along the left leg or the right leg of the patient with which the knee brace is used, and
the other end of each extension being connected respectively to one of two cuffs;
one of the two cuffs being a top cuff for at least partially attaching the knee brace to the thigh corresponding to the left knee or the right knee of the patient with which the knee brace is used, and
the other of the two cuffs being a bottom cuff for at least partially attaching the knee brace to the calf corresponding to the left knee or the right knee of the patient with which the knee brace is used;
two attachment straps including a top attachment strap for attaching the knee brace to the thigh corresponding to the left knee or the right knee of the patient with which the knee brace is used, and a bottom attachment strap for attaching the knee brace to the calf corresponding to the left knee or the right knee of the patient with which the knee brace is used;
the knee strap having an opening for positioning the knee strap around at least part of the patella of the left knee or the right knee of the patient with which the knee brace is used, and
the knee strap having dual connection straps on each of the left and right sides of the opening of the knee strap for wrapping around the back of the left knee or the right knee without hampering the popliteal crease and for securing the knee strap in position around the left knee or the right knee of the patient with which the knee brace is used; and
the knee brace being a one-size-fits-all;
whereby the knee brace is usable on the outside of the left knee or the right knee of the patient without the knee strap to unload the medial compartment of the patient's left knee or right knee with which the knee brace is used by activation of the force device to exert pressure directly by pushing at the joint line on the outside of the left knee or the right knee with which the knee brace is used,
whereby the knee brace is usable on the inside of the left knee or the right knee without the knee strap to unload the lateral compartment of the patient's left knee or right knee with which the knee brace is used by activation of the force device to exert pressure directly by pushing at the joint line on the inside of the left knee or the right knee with which the knee brace is used,
whereby the knee brace is usable on the outside of the left knee or the right knee of the patient with the knee strap to unload the lateral compartment of the patient's left knee or right knee by activation of the force device to exert pressure indirectly at the joint line on the inside of the left knee or the right knee with which the knee brace is used by pulling the knee strap wrapped around the patient's left knee or right knee with which the knee brace is used,
whereby the knee brace is usable on the inside of the left knee or the right knee of the patient with the knee strap to unload the medial compartment of the patient's left knee or right knee by activation of the force device to exert pressure indirectly at the joint line on the outside of the left knee or the right knee with which the knee brace is used by pulling the knee strap wrapped around the patient's left knee or right knee with which the knee brace is used, and
whereby the knee brace is usable on either the inside or the outside of the left knee or the right knee of the patient with or without the knee strap to provide for knee stabilization, to relieve pain from arthritis, to reduce bone on bone contact, or to compensate for decreased joint spacing in either the left knee or the right knee.

2. The knee brace of claim 1, wherein the force device comprises a selectively inflatable and deflatable air bladder that is selectively inflatable in increments.

3. The knee brace of claim 2, further comprising a display on the knee brace for displaying one or more characters representing a respective increment or increments of inflation of the air bladder.

4. The knee brace of claim 2, further comprising a pump for inflating the air bladder.

5. The knee brace of claim 4, wherein the pump is integrally formed with the knee brace.
